(19) United States
(12) Patent Application Publication
(10) Pub. No.: US 2020/0352801 A1
(43) Pub. Date: Nov. 12, 2020

(54) PORTABLE AMBULANCE WITH SITE EVALUATION

(71) Applicant: Life Preservation Technologies, LLC., New York, NY (US)

(72) Inventors: Tarek Bakkar, Beirut (LB); William W. Rutan, New York, NY (US); Frank E. Rutan, Ambler, PA (US); Marcel Botha, Brooklyn, NY (US); Arjun Kalyanpur, New York, NY (US); Ellen Crane, Brooklyn, NY (US); Salih Berk IIhan, Brooklyn, NY (US)

(73) Assignee: Life Preservation Technologies, LLC., New York, NY (US)

(21) Appl. No.: 16/965,669

(22) PCT Filed: Jan. 25, 2019
(86) PCT No.: PCT/US2019/015304

§ 371 Date: Jul. 29, 2020

Related U.S. Application Data
(60) Provisional application No. 62/628,878, filed on Feb. 9, 2018.

(51) Int. Cl. A61F 17/00; A45C 15/06; A45C 11/00; A45C 13/02; A45F 3/04; H04N 5/225; G08B 7/06

(52) U.S. Cl. CPC A61F 17/00; A45C 15/06; A45C 11/00; A45F 2003/003; A45F 3/04; H04N 5/2253; G08B 7/06; A45C 13/02

(57) ABSTRACT

A portable ambulance for use at a multi-casualty incident site including a siren for crowd parting, at least one flashing light mounted to a portable medical equipment carrying case, and a camera supported by the carrying case. The carrying case is removable from a vertical position on the wearer and placed in a horizontal position wherein one or more panels is openable to display the medical supplies contained within the case. The camera and at least one flashing light are supported on an extendable pole movable from a retracted position to an extended position. Microcontrollers in the case control the camera and flashing light.

This application claims priority to provisional application 62/628,878, filed Feb. 9, 2018, the entire contents of which are incorporated herein by reference.

### BACKGROUND OF THE INVENTION

### 1. Field of the Invention

This application relates to a portable ambulance, and more particularly, to a portable ambulance transportable by a first responder to multiple casualty incidents and including site evaluation capabilities.

### 2. Background of the Related Art

The DHS Report, “Project Responder 4: 2014 National Technology Plan for Emergency Response to Catastrophic Incidents” lists numerous key capabilities/areas that could benefit from technological innovation. This list illustrates the deficiencies in current responses/responders to mass (multiple) casualty incidents (MCI's) and includes: 1) situational awareness, 2) communications; 3) command, control and coordination; 4) responder health, safety and performance; 5) logistics and resource management; 6) casualty management; and 7) training and exercise.

Situational awareness requires the ability to communicate with responders and peers as well as knowledge of their location and their proximity to risks and hazards in real time. This includes the ability to rapidly identify hazardous agents and contaminants at the incident site. Situational awareness would also benefit from the capability of incorporating information from multiple and nontraditional sources, e.g., crowdsourcing and social media, into incident command operations. Currently, such effective situational awareness at MCI's is lacking.

Communications involves the ability to communicate with responders in various environmental conditions, including through barriers, inside buildings, underground, etc. Currently, during an incident, communication gets confusing, especially in terms of where the responders/EMS personnel should go. Communicating which station is for which hospital and ambulances is difficult and sometimes important information and communication gets missed. Additionally, responders frequently rely on a two way radio for communication which is limiting as oftentimes 30+ people are talking so there is communication chaos. Sometimes communication breaks down due to many stakeholders and actors simultaneously making calls over the radio. With insufficient communication, valuable treatment time for the injured is lost, resulting in unnecessary loss of lives, increased injuries and long term health consequences. Currently, such efficient and reliable communications are lacking at incident sites.

Command, control and coordination include the ability to remotely monitor the tactical actions and progress of all responders involved in the incident in real time. It also involves a centralized control to properly coordinate location and tasks of first responders. The ability to identify trends, patterns and important content from large volumes of information from multiple sources at the site, including nontraditional sources, are beneficial to support incident decision making. It also includes the ability to identify, assess and validate emergency response related software applications. Currently, such command, control and coordination at MCI's are in need of improvement.

Responder health, safety and performance include providing the first responders with protective clothing and equipment that protects against multiple hazards. For example, in many instances, EMS professionals follow behind the first responders because they lack Kevlar bulletproof vests. This causes delay in treatment of injured people at the incident. Moreover, oftentimes after such incident, such as a terrorist attack, there are hazardous materials which pose a health risk to first responders. Additionally, sometimes at the site of a terrorist attack, there is a planned secondary event, e.g., a second explosion or other attack, which leaves the first responders vulnerable. Therefore, there is a need to protect the safety of the first responders. Additionally, protecting the safety would speed up treatment of the injured as the first responders can focus solely on treating the injured without taking time consuming precautions which would otherwise be necessary.

Logistic and resource management include the ability to identify what resources are available to support a response (including resources not traditionally involved in a response), what their capabilities are, and where they are in real time. It also includes the ability to monitor in real time the status of resources and their functionality. Tied into resource management is the need for efficient organization of first responder bags carrying medical equipment. Currently, EMT bags are confusing for several reasons: 1) they are lots of different types of equipment so bags can differ; 2) the bags contain multiple compartments so finding the desired equipment can be difficult and time consuming; and 3) the bags are not properly organized so as first responders search through the bags to locate desired items, the contents become even more disorganized and haphazard, further increasing the difficulty of finding the necessary medical treatment items. Thus, valuable time is lost resulting in loss of life or increased injuries as a result of delayed treatment.

Casualty management includes the ability to remotely scan an incident scene for signs of life and decomposition to identify and differentiate casualties and fatalities. The need exists for improvements to such current management techniques.

As can be appreciated, during such Multi Casualty Incidents (MCI'S) such as terror attacks, e.g., gas attacks, bombings, etc., multi-car crashes, fires, natural disasters, etc., it is imperative that the first responders gain access to the injured as fast as possible. This oftentimes involves having to pass through dense and chaotic crowds. Additionally, quick and easy access to the necessary medical equipment is essential. This need becomes more critical in triage conditions which often accompany such incidents. MCI's are characterized by volatile and messy conditions in which current bags may break down, become contaminated and require intense cleaning. As noted above, currently, first responder bags differ so the first responder wastes valuable time in trying to locate within the particular bag the necessary supplies. Therefore, the need exists for quick and efficient access to desired medical equipment, not only at the outset but during the entire time at the incident site to reduce the chances of the bag contents easily become disorganized and hard to locate which can negatively affect optimal care. Still further, portability, e.g., compact and light weight bag and equipment, is beneficial to reduce fatigue of the first responders. With the stress and intensity of the treatment of the injured, it would be beneficial if the first responder did not expend unnecessary energy in movement, i.e., transport of the bag and supplies, from person to person for treatment. However, the need for light weight needs to be balanced with the necessity of the responders having in their possession a sufficient amount of medical equipment, i.e., more than what they might need for any situation because the needs can vary from incident to incident and such needs are not fully known prior to arrival at the incident site. MCI's are spontaneous and varied events ranging from natural disasters to terrorist attacks, each with their own specific medical equipment requirements. It would be advantageous to provide a more modular system that is adaptable to these varied situations. That is, since needs at various situations differ, the first responders need to be prepared for any situation which requires provision of more equipment; however, this need must be balanced with the disadvantage of carrying heavy weight bags which leads to fatigue over time. Also, unbalanced bags can lead to fatigue and wear and tear on first responders after long periods of time, leading to great inefficiency. Therefore, a balance needs to be effectively achieved between supplying sufficient equipment for preparation for any type of emergency while not unduly increasing the weight of the equipment. Also of note is that prior carrying equipment has attempted without success to meet the foregoing criteria. Certain prior art bags provide some of the features, but at the expense of other features, or are missing the critical aspects discussed herein.

As noted above, the first responders are put in a range of situations where their personal safety is at risk by other factors such as environmental conditions or another unpredictable secondary event such as a second terror attack. Therefore, it would be advantageous to provide sufficient protection for the first responders to reduce their vulnerability which would not only protect the health and safety of the first responders but increase efficiency as they can focus on treatment of the injured with less distraction/fear of the situation.

Additionally, documentation is critical to triage and transporting patients safely, but manually done, it requires too much time given the severity and chaos of MCI's. As much, if not more, insights are gleaned from personnel being able to look back upon an incident to see what occurred and actions taken. Therefore, the need exists for improved information gathering.

In summary, the need exists to provide first responders with equipment that meets the foregoing criteria, thereby improving response to MCI's by first responders. Thus, it would be advantageous to transform the first responder to what is essentially a “portable ambulance.” It addition to the foregoing, it would also be beneficial to provide an enhanced portable ambulance which has site evaluation capabilities which includes data collection. Site evaluation capabilities would advantageously gather information in real time for analysis by the first responder as well as by incident coordinators and law enforcement officials. With the main priority at the incident being to treat the injured, the focus is not on preservation of evidence as the rush is for medical treatment. Therefore, important evidence can be lost or destroyed at the scene of the incident. Thus, it would be beneficial to enable information gathering without adversely affecting treatment of the injured.

Thus, by providing an enhanced “portable ambulance,” the first responder could be advantageously provided with equipment which provides quick access through dense crowds to the surgical site, enables information gathering and data collection at the site and surrounding area, protects the first responder from personal injury, and enables easy access to selective medical equipment. It would be advantageous to provide the foregoing in an easily transportable carrying bag that reduces first responder fatigue and allows rapid movement from person to person. Currently, no system effectively achieves this.

### SUMMARY

The present invention overcomes the disadvantages and deficiencies of the prior art. The present invention advantageously facilitates access to victims in mass casualty incidents (MCI's) and provides the first responder with easy access to and transport of medical equipment for treating the victims, while protecting the first responder and collecting data at the scene. In some embodiments, it can allow real time communication with a command control center. Rapid response translates to saving lives, and the system of the present invention, by enabling clearing through crowds, efficient organization of personnel and equipment and coordinated communication improves speed of treatment. Further, the system of the present invention not only saves lives of the victims and protects the health and lives of first responders, but simultaneously enables gathering data at the scene which can be used for later analysis and can potentially be utilized to prevent further incidents. Thus, in essence, the present invention provides a safer, cost effective and highly efficient “portable ambulance” for MCI's. Each of the features and the attendant advantages are discussed in detail below.

In accordance with one aspect of the present invention, a portable ambulance is provided comprising a siren for crowd parting, a flashing light mounted to an extendable pole, and a portable medical equipment carrying case, wherein the extendable pole is mounted to the carrying case and extendable with respect to the carrying case. A camera is mounted to the extendable pole, and the extendable pole is movable from a retracted position wherein the camera is closer to the case and an extended position wherein the camera is further from the case.

In some embodiments, the system includes an activation switch that enables activation of the siren and/or light. In some embodiments, the system includes an activation switch wherein turning on the activation switch automatically turns on the camera. In some embodiments, the system includes a flashing light on the case, wherein when the case is opened the flashing light automatically changes to a static light or turns off. In some embodiments, the first and second openable panels of the case include a Velcro surface to hold medical supplies. In some embodiments, the case has internal lighting for viewing contents at night.

In accordance with another aspect of the present invention, a portable ambulance is provided comprising at least one blinking light, a siren and a portable medical equipment carrying case, the flashing light and siren attached to the carrying case. The carrying case has a) a vertical position for wearing by a user on a back of the user and b) a horizontal position when removed from the wearer, wherein in the horizontal position a rear panel of the carrying case is positionable parallel to a support surface, and a front panel is pivotable to an open position transverse to the rear panel and a side panel is pivotable to a position more aligned with the rear panel. Upon opening the case, the at least one blinking light automatically changes its blinking status.

In some embodiments, the status of the blinking light changes to a static light; in other embodiments, the status of the blinking light changes to an off position. In some embodiments, upon closing the front and side panels, the light automatically reverts back to its blinking status.

In some embodiments, the front and/or side panel is composed of multilayered materials including a plastic interposed between two layers of nylon material.

In some embodiments, the portable ambulance includes a beacon having an extendable pole, extendable with respect to the carrying case, and a flashing light and camera are positioned at a top region of the extendable pole. In some embodiments, the pole is pivotable from a vertical position to a horizontal position.

In accordance with another aspect of the present invention, a portable ambulance for treating patients at a multi-casualty incident site and simultaneously passively gathering data is provided, the portable ambulance comprising at least one blinking light, a siren, a surveillance camera and a light weight portable medical equipment carrying case. The flashing light and siren are attached to the carrying case which has a) a vertical position for wearing by a user on a back of the user and b) a horizontal position removed from the wearer, the carrying case having an openable first panel and an openable second panel, wherein the first and second panels are composed of multi-layers of material.

In some embodiments, the first and second panels have a Velcro surface on an interior to hold medical supplies. In some embodiments, in the horizontal position, the first and second panels are openable in opposite directions to display medical supplies contained within the carrying case.

In accordance with another aspect of the present invention, a transportable carrying case for first responders for treating patients at a multi-casualty incident site and simultaneously passively gathering data is provided, the carrying case comprising at least one blinking light, a siren, a camera, a first strap for supporting the case on a wearer's back and a second strap extending over at least a portion of the first strap, the second strap supporting an actuator to actuate the siren and light.

In some embodiments, the second strap includes a channel providing a passage for wires from the actuator, the wires extending thought the channel into the carrying case such that the wires are independent of the first strap. In some embodiments, a flap is provided on a rear of the strap, the flap openable to access the wires.

In accordance with another aspect of the present invention, a vest wearable by a first responder at a multi-casualty incident is provided, the vest comprising an LED, an air quality sensing unit for measuring levels of one or more gasses, and a display viewable by the first responder while the vest is worn by the first responder. In some embodiments, the air quality sensor is contained in a hard shell pouch that attaches to the vest, thus once attached becoming part of the vest. The vest further includes a microcontroller in communication with sensors of the air quality sensing unit to receive first signals from the sensors and transmit second signals in response to the received signals to provide a readout of gas levels to alert the first responder of hazardous air conditions, the gas levels further being stored for future reference. The gas levels are transmitted to a remote site. In some embodiments, the vest further includes a pouch containing medical supplies, the pouch removably attachable to the vest. The pouch in some embodiments has a front cover hingedly attached at a bottom portion to the pouch for pivoting movement to an open position for viewing and accessing the supplies within the pouch while wearing the vest. In some embodiments, the display is positioned above the pouch.

In accordance with another aspect of the present invention, a system for first responders for use at a multi-casualty incident is provided, the system comprising a) a backpack worn on a back of the first responder, the backpack including a siren, a flashing light and a camera for taking and storing images at a site of the multi-casualty incident, the backpack having at least one openable panel to display medical supplies carried within the backpack and having a weight of about 25 pounds when empty (without medical supplies) and a weight of about 45 pounds when full with medical supplies; and b) a vest worn on a chest of the first responder, the vest being bulletproof and including an air quality sensor for measuring gas levels at the site.

### BRIEF DESCRIPTION OF THE DRAWINGS

So that those having ordinary skill in the art to which the subject invention appertains will more readily understand how to make and use the surgical apparatus disclosed herein, preferred embodiments thereof will be described in detail hereinbelow with reference to the drawings, wherein:

FIG. 1A is a flow chart depicting a first embodiment of the system of the present invention;

FIG. 1B is a flow chart depicting the electronics of the system of FIG. 1A;

FIG. 1C is a flow chart depicting an alternate embodiment of the system of the present invention;

FIG. 1D is a table of the elements/features of the flow chart of FIG. 1A;

FIG. 1E is a table of the elements/features of the flow chart of FIG. 1C;

FIG. 2 is a front view of a first embodiment of a smart bag of the present invention, the beacon pole shown in the extended position;

FIG. 3 is a bottom perspective view of the smart bag of FIG. 2;

FIG. 4 is a rear perspective view of the smart bag of FIG. 2 showing the rear straps;

FIG. 5 is a close up view of the top portion of the smart bag of FIG. 2 showing the carrying handle;

FIG. 6 is a close up view of the top portion of the smart bag of FIG. 2 showing the rotation locking mechanism for the pole;

FIG. 7 is a front view of the smart bag of FIG. 2;

FIG. 8 is a side view of the smart bag of FIG. 2;

FIGS. 9 and 10 are perspective views of the smart bag of FIG. 2 in the horizontal position with the front and side flaps (panels) open to show the stored contents;

FIG. 11A is an exploded view of the smart bag of FIG. 2;

FIG. 11B is an exploded view of the rear frame support of FIG. 11A;

FIG. 11C is a perspective view of the assembled rear frame support of FIG. 11B;

FIG. 12A is a front view of the front panel of the smart bag of FIG. 2;

FIG. 12B is an exploded view of the front panel of FIG. 12A;

FIG. 12C is a side view of the front panel of FIG. 12A and FIG. 12D is a side perspective view from the other side of the front panel of FIG. 12A;

FIG. 13A is a front perspective view of the left side panel (containing the clasp) of the smart bag of FIG. 2;

FIG. 13B is a rear view of the left side panel of FIG. 13A showing the inner side;

FIG. 13C is an exploded view of the left side panel of FIG. 13A;

FIG. 13D is a side view of the front panel of FIG. 13A and FIG. 13E is a side view of the other side of the front panel of FIG. 13A;

FIG. 14A is side perspective view of the right side panel of the smart bag of FIG. 2;

FIG. 14B is an exploded view of the front panel of FIG. 14A;

FIG. 14C is a side view of the front panel of FIG. 14A;

FIG. 15 is a perspective view of the assembled smart bag of FIG. 11A;

FIG. 16 is a view from one side (the left side) of the assembled smart bag of FIG. 11A and FIG. 17 is a side view from the opposing side (the right side);

FIG. 18 is a cross-sectional view taken along line A-A of FIG. 17 to show internal components of the bag;

FIG. 19 is a front view of the assembled smart bag of FIG. 11A;

FIG. 20 is a top view of the assembled smart bag of FIG. 11A;

FIG. 21 is a side view of the strap of the smart bag of FIG. 2;

FIG. 22 is a schematic side view showing attachment of the strap of FIG. 21 to the smart bag and showing the secondary strap and channel for the cable;

FIG. 23A is a perspective of the button housing on the strap of FIG. 21;

FIG. 23B is a side view of the button housing of FIG. 23A;

FIG. 23C is a rear view of the button housing on FIG. 23A;

FIG. 23D is a cross-sectional view taken along line D-D of FIG. 23A;

FIG. 23E is a cross-sectional view taken along line E-E of FIG. 23A;

FIG. 24 is a schematic view of a front portion of the secondary strap and the button housing;

FIG. 25 is a schematic view of the rear portion of the strap of FIG. 24 showing the flap in the open position;

FIG. 26 is a side view of the strap of FIG. 24;

FIG. 27A is a side view of the one embodiment of the beacon of the present invention showing the extendable pole (support) in the retracted position;

FIG. 27B is a side view of the beacon of FIG. 27A showing the pole in the extended and vertical position;

FIG. 27C is a side view of the beacon of FIG. 27A showing the pole in the extended and horizontal position;

FIG. 28 is an exploded view of the beacon of FIG. 27A;

FIGS. 29A and 29B are longitudinal cross-sectional views of the beacon of FIG. 28 in the retracted, non-rotated position;

FIG. 30A is a perspective view of the rotator of the rotation assembly of the beacon of FIG. 28;

FIGS. 30B and 30C are perspective and front views, respectively, of the fixed connector of the rotation assembly of the beacon of FIG. 28;

FIG. 30D is a perspective view of the rotation key of the rotation assembly of the beacon of FIG. 28;

FIG. 30E is a top view of the rotation key of FIG. 30D;

FIG. 30F is a perspective view of the release base of the rotation assembly of the beacon of FIG. 28;

FIG. 31A is a perspective view of the boom base of the beacon of FIG. 28;

FIG. 31B is a perspective view of the boom lock of the beacon of FIG. 28;

FIG. 32 is a front perspective view of one embodiment of the vest of the present invention showing the front pouch in the closed position;

FIG. 33 is a front perspective view of the vest of FIG. 32 showing the front pouch in the open position;

FIG. 34 is a top perspective view showing the contents of the front pouch of FIG. 33;

FIG. 35 is a front perspective view of the vest of FIG. 32 with the pouch removed;

FIGS. 36A, 36B and 36C illustrate respectively an embodiment of the 1) vest; b) vest and smart bag; and c) vest, smart bag and helmet of the systems of the present invention;

FIG. 37A is a perspective view of an alternate embodiment of the smart bag of the present invention shown on a wearer's back;

FIG. 37B is a perspective view showing the smart bag of FIG. 37A seated horizontally on the ground and showing the flaps open to display the internal contents;

FIG. 37C illustrates the smart bag of FIG. 37A seated horizontally on the ground adjacent an injured person;

FIG. 37D is a perspective view of the front portion of a vest for use with the bag of FIG. 37A, the vest shown on a wearer;

FIG. 38A is a perspective view of another alternate embodiment of the smart bag of the present invention shown on a wearer's back;

FIG. 38B is a perspective view showing the smart bag of FIG. 38A on the ground vertically with the flaps open to display the internal contents;

FIG. 38C is a perspective view of the front portion of the vest for use with the bag of FIG. 38A, the vest shown on a wearer;

FIG. 39A is a perspective view of another alternate embodiment of the smart bag of the present invention shown on a wearer's back;

FIG. 39B is a perspective view showing the smart bag of FIG. 39A on the ground horizontally with the flaps open to display the internal contents;

FIG. 39C is a side view illustrating the smart bag of FIG. 39A on the ground in the respective closed, half open and fully open positions; and

FIG. 39D is a perspective view of the front portion of the vest for use with the bag of FIG. 39A, the vest shown on a wearer.

### DETAILED DESCRIPTION OF PREFERRED EMBODIMENTS

For a full understanding of the present invention and its attendant advantages, an understanding of the chaos at a Mass Casualty Incident (MCI) and the challenges and risks of first responders will first be discussed. An MCI essentially has three time components. The first component, i.e., the beginning, is what happens before the first responders arrive. The second component, i.e., the middle, is the actual response including triage, patient assessment, treatment, and transport of individuals from the scene to hospitals or other venues. The third or last component is the resolution of the MCI and the demobilization of the first responders and EMS professionals. During the middle component, when the first responders arrive, speed is of the essence, both in access to the injured as well as in treatment of the injured. It is recognized that fast/easy access to medical equipment helps in speedier treatment of the injured. The present invention, as will be discussed in detail below, enables such quicker and efficient access and treatment.

In addition to the three foregoing components, a post analysis/evaluation of the scenario is also beneficial. The present invention also provides improved ways to assist such post incident analysis. Analysis of the MCI can be beneficial for several reasons: 1) to analyze the first responders actions to determine where improvements can be made in future responses to MCI's; 2) to analyze the site to determine steps which can be taken to prevent future attacks; and 3) to analyze the site if it is a result of a human-induced event such as terrorist attack to locate the perpetrators (analogous to a crime scene investigation). In arriving at the scene, the focus of the first responders is rightfully on life saving steps and not evidence preservation and therefore unfortunately evidence can be lost if not collected initially as the first responders rapidly work to clear objects, move the injured, etc. However, the present invention provides a way for the first responders, who are usually the first to arrive, to passively collect data (without distracting from the treatment efforts) to be shared with later arriving law enforcement officials before evidence is destroyed by the focus on triage. This is discussed in more detail below.

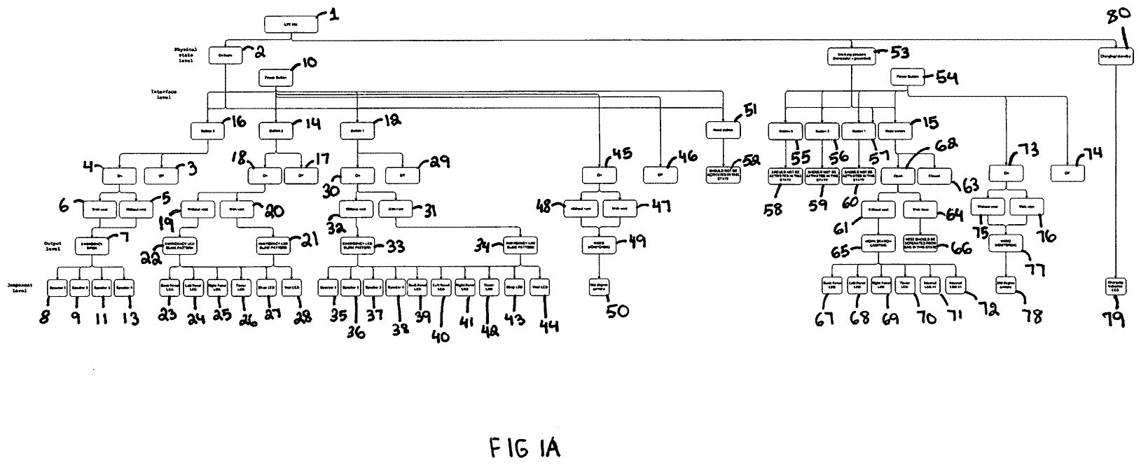

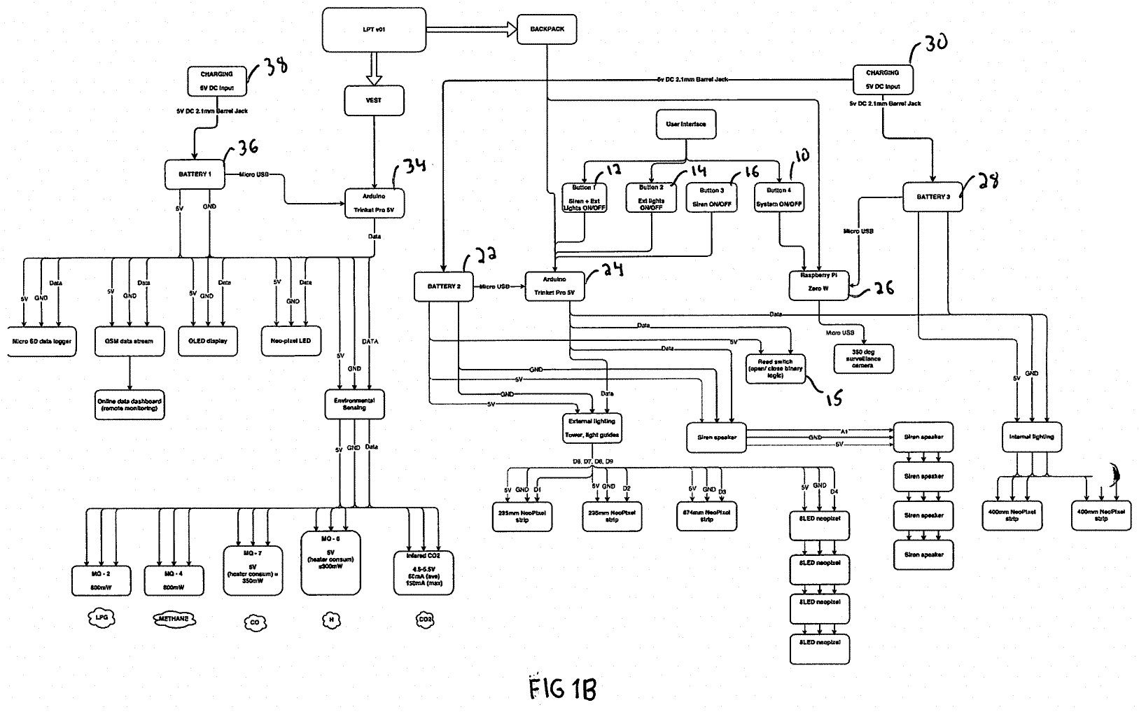

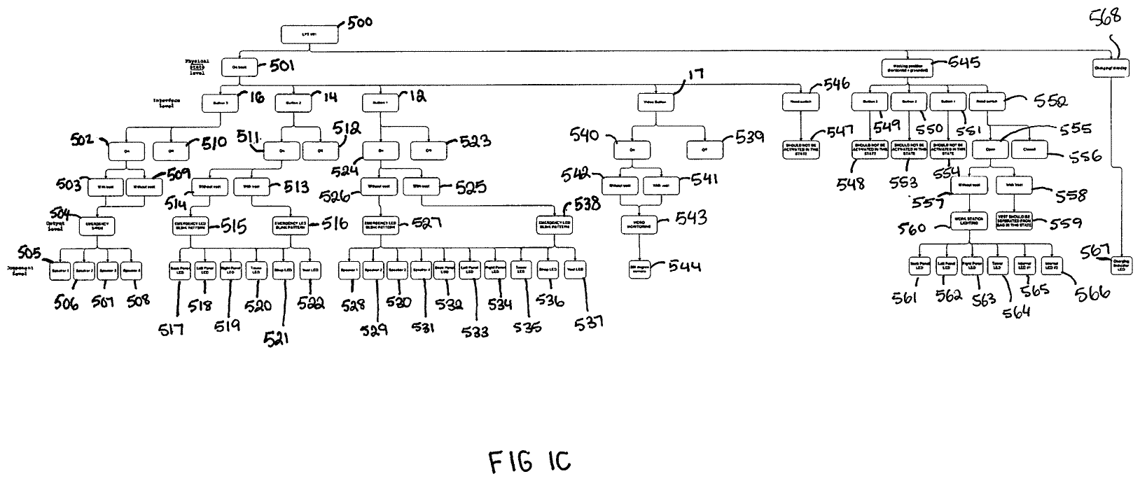

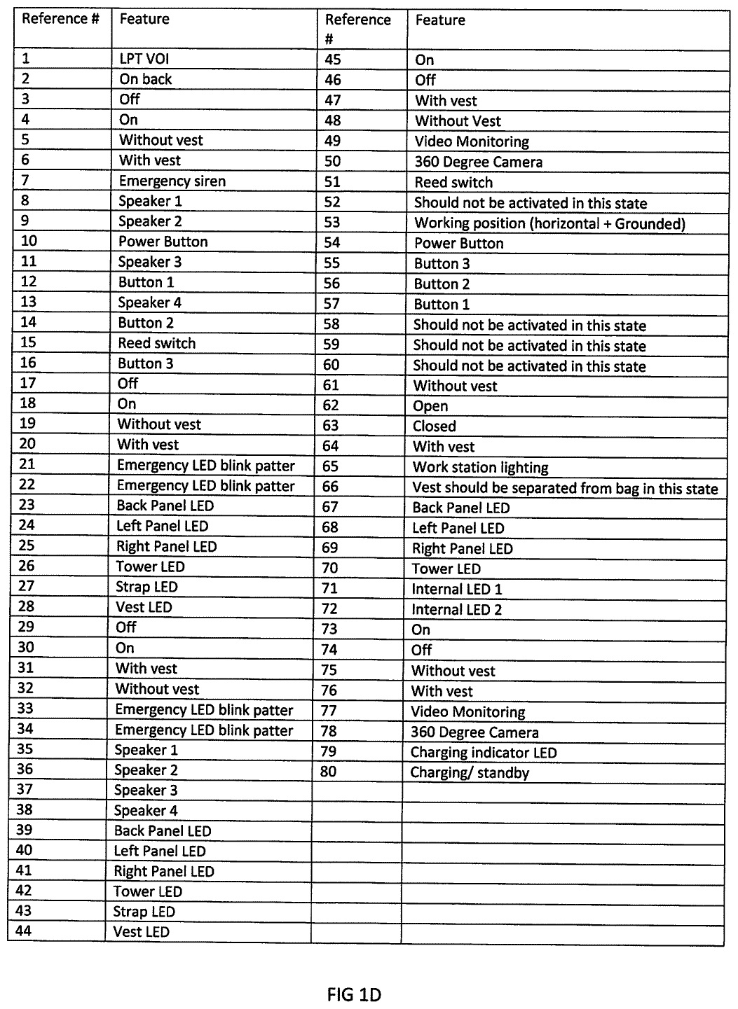

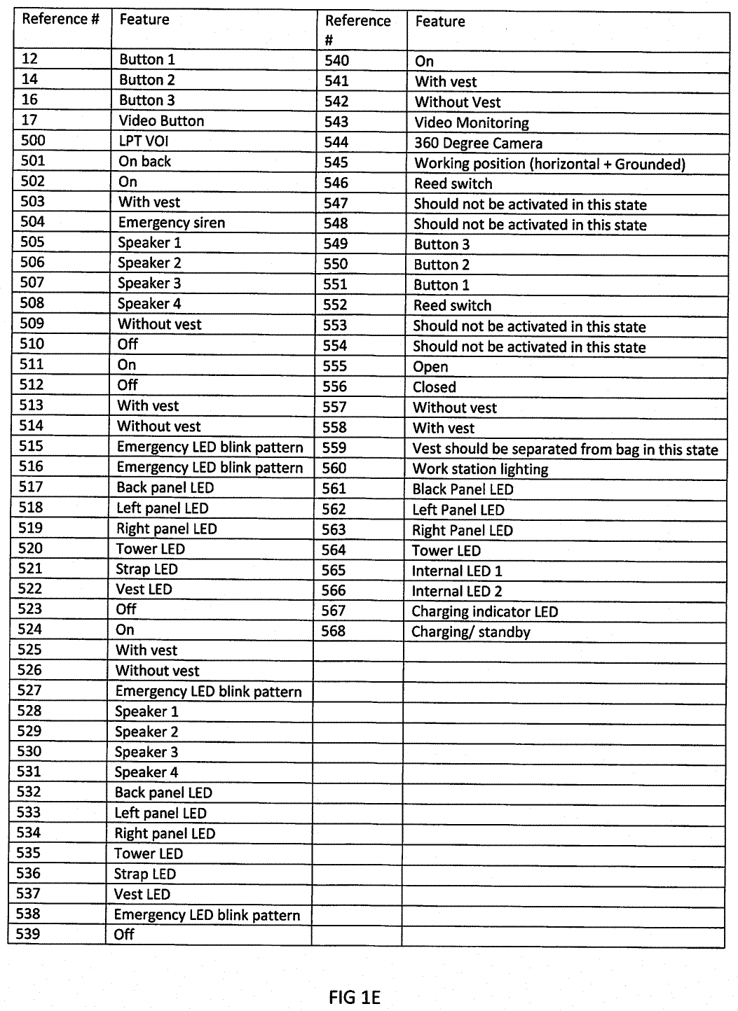

Referring now to the drawings wherein like reference numerals identify similar structural features of the device disclosed herein, FIGS. 1A and 1B provide flow charts illustrating the components and function of the portable ambulance of the present invention. FIG. 1D is a table of the elements/features of the flow chart of FIG. 1A; FIG. 1E is a table of the elements/features of the flow chart of FIG. 1C. At the outset, it should be appreciated that the present invention includes a) a shoulder worn bag, which can be in the form of a backpack-like device; and b) a vest. The bag and vest each have separate electronics and work independently of each other. Thus, in some embodiments only the bag or only the vest is worn and used. However, in some embodiments it is contemplated that both the vest and bag/backpack can be worn and used together. This can be appreciated in FIGS. 36A-36C wherein FIG. 36A illustrates the vest alone (e.g., vest 300) of the present invention being worn by a user, e.g., a first responder, and FIG. 36B illustrates the bag (e.g., bag 100) of the present invention and the vest 300 being worn by a user. In FIG. 36C, the wearer also has a helmet 330. Details of the various bag and vest configurations and internal components will be discussed in detail below.

Turning back to the flow charts, FIG. 1A provides an overview of the system and the user interface, and FIG. 1B provides a circuit diagram. The diagram of FIG. 1A encompasses the bag and vest, even though they are independent, and each will be discussed separately.

Viewing the smart bag (portable medical equipment (supply) carrying case) first, the smart bag includes a system power button 10 on the exterior of the bag which turns the system on and off. When the switch 10 is activated, it initiates the electronics so the siren and lights can be activated and also turns on the video monitoring, i.e., the 360 degree camera. In this way, the wearer/user (first responder) does not have control over the surveillance since it is automatically activated when the system is activated. Also, the automatic activation means the wearer/user does not have to take the additional step of turning on the surveillance camera and thus not only avoids the distraction of activating the camera amid the chaos at the scene but avoids the possibility of the user mistakenly forgetting to activate the surveillance camera, thereby forgoing the advantages of the surveillance system as discussed herein such as loss of evidence, reduced evaluation of the scene, etc. Note in the alternate embodiment of the system of FIG. 1C, an initial switch does not need to be actuated in order to turn on the siren and lights (unlike the system of FIG. 1B where first the system is turned on and then the user can turn on siren and lights). In FIG. 1C there is a separate button, denoted as the video button 17, to turn on the surveillance camera. In all other respects, the system of FIG. 1C is identical to the system of FIG. 1A so the description of FIG. 1A and the electronics of FIG. 1B are fully applicable to system of FIG. 1C. In another embodiment, a system power button is on the bag to turn on the power and the camera and there are two buttons on the strap of the bag—one to turn the lights on and off and one to turn the siren on and off.

With continued reference to FIG. 1A, once the system is actuated by turning on the switch 10, the wearer (first responder) has the option of activating one of three buttons: 1) button 12 which activates both a flashing LED and an emergency alarm/siren; 2) button 14 which activates just an emergency flashing (blinking) LED; and 3) button 16 which activates just the alarm/siren. Preferably the alarm/siren simulates a standard ambulance siren so people are warned of an approaching “ambulance” to clear the space for the first responder. In one embodiment, the bag has four speakers for the siren, identified in the flow chart as speakers 1, 2, 3 and 4. The speakers in preferred embodiments can be located in the bottom of the bag. Although four speakers are provided, a different number of speakers and/or different locations of the speakers can be provided. In one embodiment, the LED is a blinking light and five lighted regions are provided, one in each of the back panel, left panel, right panel, tower (beacon) and strap of the bag. It is also contemplated that a different number of LED lights and/or different locations can be provided. The LED's in the panels can be provided in light guides, with several LED lights spaced apart along the guide.

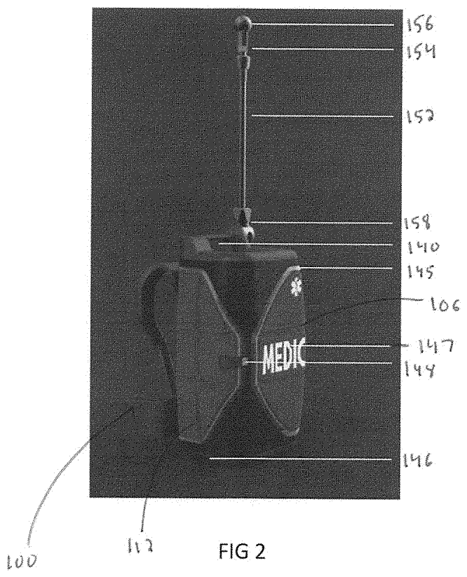

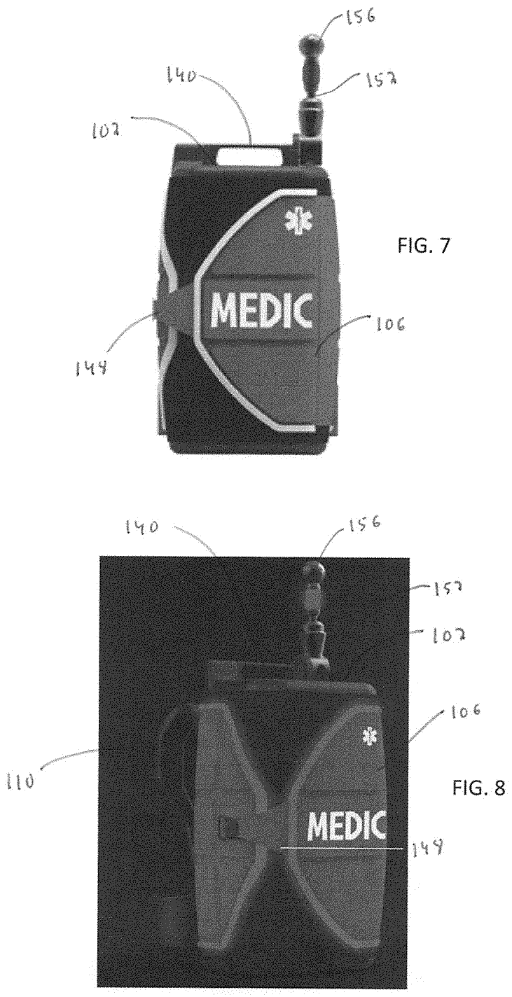

The bag, as will be discussed below, has an upright or vertical position and a horizontal position. The vertical position corresponds to the position worn by the user as shown in FIG. 36A. This upright position is also shown for example in FIGS. 2 and 7. In the horizontal position, the bag is opened as discussed below so the wearer can access the contents within the bag. Thus, as can be appreciated from the flow chart of FIG. 1A, when the bag is on the ground in its horizontal position and opened in a tool-box like manner, a reed (magnetic) switch 15 is automatically activated to change the flashing LED lights to static LEDs, e.g., constant white light. More specifically, in one embodiment the front panel has a magnet and the top cap of the bag has a corresponding magnet in their interiors. When the front panel is open, it opens the circuit and changes the voltage, thereby turning on the interior ambient (work station) lights of the bag. In some embodiments, the ambient lights are positioned in the openable front panel and left side panels, discussed in more detail below. The reed switch is also wired to the exterior LEDS in the panels and beacon (tower) to effect their transition from the blinking light. This automatic transition facilitates visualization of the contents of the bag since the interior of the bag is lit with constant light without the distraction of the flashing light. The flow chart refers to these static LEDs as internal LED #1 and internal LED #2 which as discussed below, in some embodiments include two LED strips in the interior of the bag, one on the front panel and one on the side panel. When the bag is closed, the reed switch automatically transitions the static lights back to the flashing LED and turns off the internal LEDs. This facilitates the first responder moving through crowds to treat another victim since the flashing light provides a warning signal for spectators/non-essential personnel to disperse/clear.

Note the four LEDs (back panel, left panel, right panel and tower) are in the aforedescribed embodiment designed to transition to static light when the bag is removed from the wearer's back, placed in the “working position” (horizontal on the ground) and opened. However, it is also contemplated that the LEDs alternatively can be shut off when the bag is opened. It is further contemplated as another alternative that some of the LEDs are turned off, while others remain on, either static or blinking, such as the tower LED.

Note as depicted in the flow chart, buttons 12, 14 and 16 which turn on the flashing lights and siren are intended not to be activated by the first responder when the bag is in the horizontal working position. In some embodiments, the lights and siren can be wired so that the buttons 12, 14 and 16 cannot be actuated (even if attempted) if the bag is in the working (horizontal) position and/or the bag is in the working position with the flaps open.

The bag also includes a charging capability utilized when the bag is in an upright position such as in a locker or standing in a vertical position before use. The charging receptacle can be plugged into a standard electrical outlet. A static LED can be provided to indicate that the electronics of the bag are being charged. Alternatively, this could be achieved by programming LED strips to glow when being charged, rather than a separate LED.

The flow chart of FIG. 1A also helps to explain the electronics of the vest. When the on/off switch on the vest is turned on, the electronics is activated and the wearer can activate a button on the strap of the vest to turn on a blinking LED. In alternate embodiments, the vest does not need to be initially actuated and the wearer can initially actuate the LED button without turning on the on-off switch. In preferred embodiments, the LED is located on the strap of the vest, however, it is also contemplated that it can be located on other regions of the vest and also contemplated that more than one LED can be provided on the strap of the vest or other regions of the vest. The vest in the flow chart has an LED but not a siren. However, the vest can in some embodiments include a siren or alarm. As indicated in the flow chart, the vest is preferably separated from the bag in the working state of the bag.

The vest can also include an air quality sensor. This sensor can be activated upon activation of the on/off switch or alternatively configured to be enabled when the activation switch is turned on, but requires subsequent action by the wearer to separately turn on the air quality sensor. The former is illustrated in the flow chart of FIG. 1B as the sensor button activates the air quality sensor. Although it is contemplated that only the vest has an air quality sensor, in alternate embodiments, the bag can have an air quality sensor and a corresponding display to indicate measured levels. Also, the air quality sensor can alternatively be contained in a hard shell pouch seating unit that attaches to the vest.

The electronics of the systems of the present invention will now be discussed in conjunction with FIG. 1B. Turning to the smart bag, i.e., backpack, first, the system includes a battery 22, e.g., a 5V battery, connected to the external and tower lights within light guides. These lights in this embodiment are in the form of three separate strips which are carried by the front panel, left side panel and right side panel of the bag, denoted as neopixel strips on the flow chart. The beacon also includes a series of four light strips, each containing one or more LEDs, denoted as LED neopixel on the flow chart. These lights can be connected in series in a 360 degree pattern. More details of these lights are discussed below. Note that in the preferred embodiment, the lights utilized with the system are LEDS, however, it is also contemplated that other types of lighting can be utilized. The lights are also electrically connected to a microcontroller 24 such as the depicted Arduino Trinket Pro 5V. The controller 24 enables control of the color, blinking pattern, etc. of the lights. A different number of lights and different locations and configurations are also contemplated. The battery 22 and controller 24 are also wired to the siren speakers, four of which are depicted in the illustrated embodiment, although a different number of speakers can be utilized. The speakers are also connected to the controller 24 so the controller can provide, for example, variable tones. Preferably the tones are preset in the microcontroller code.

Controller 24 also is also connected to the reed switch for control of the lighting. More specifically, the reed switch 15, embodying an open/close binary logic, acts like a sensor wherein when the bag is opened, a pulse voltage signal is sent to the controller 24 which then sends a signal to the interior lights to turn on the interior lights on the front and rear panels, or alternatively lights on the other panels. When the bag is closed, the reed switch 15 sends a signal to the controller 24 which in response sends a signal to turn off the interior lights. The interior lights are denoted as 400 mm neopixel strips on the FIG. 1B flow chart (and in the flow chart of FIG. 1A as internal LED #1 and LED #2).

Controller 26, such as the Rasberry Pi Zero W, is electrically connected to the surveillance camera to control the camera and enable data to be transmitted to a central command remote from the wearer. A second battery 28 in the bag provides power to the controller 26. Battery 28 is also wired to the internal lighting within the bag, e.g., the light strip on the front and rear panels of the bag.

Further depicted in the flow chart of FIG. 1B is the user interface providing the four buttons discussed above: buttons 10, 12, 14, and 16 to turn on the system/surveillance camera, LED and siren, LED alone or siren alone, respectively. Button 10 as shown is in communication with controller 26 and buttons 12, 14 and 16 are in communication with controller 24.

A charging 5V DC input port 30 is utilized to charge both batteries 22 and 28 and can be plugged in via a cable to a standard electrical outlet.

Turning now to the electronics of the vest and with continued reference to the flow chart of FIG. 1B, the vest includes a microcontroller 34, e.g., an Arduino Trinket Pro 5V. As can be appreciated, the vest has its own electronics, e.g., its own microprocessor, operable independent of the electronics, e.g., microprocessor, of the bag. More specifically, the microcontroller 34 is connected to an environmental sensing unit to evaluate air quality at the MCI, thus providing an air quality readout of the ambient environment. The air quality sensor can measure various gasses in the surrounding environment with one example depicted in the flow chart of FIG. 1B. In this example, sensor(s) can measure LPG, methane, carbon monoxide (CO), hydrogen (H) and carbon dioxide (CO2) to determine if hazardous conditions are present, thereby protecting the first responder. Various sensors can be utilized to measure these gasses or other gasses in the atmosphere. The vest also includes a Micro SD data logger which provides local storage of data obtained from the sensors which is pushed to the web host dashboard on the vest, thereby providing a display panel of the data obtained by the gas measurements. Further provided is the GSM data stream to push data to the on line dashboard. This provides both a display of gas readings for the wearer as well as transfer of data to a remote site for monitoring so the first responder can be alerted by the remote command center if hazardous gas conditions are present or adversely change over time at the MCI. The vest dashboard can include an OLED display, located in preferred embodiments on a top portion of the vest, to indicate measured values of gas so the wearer can assess the air quality to determine any risks. That is, the OLED display provides air quality readings to the user, e.g., provides readings of the data/measurement data to the user obtained from the gas sensors. The display in some embodiments is on the top portion of the vest, easily visible by the user while wearing the vest. In this manner, when the wearer (first responder) approaches the site, the wearer can turn on the sensors and quickly evaluate the safety of the environment. The system can also include a visual or audible alert triggered if a measured gas reading exceeds a predetermined threshold.

Also included in the vest is a blinking (flashing) LED, (denoted in the flow chart as neo-pixel LED) preferably wired to the controller 34 so the flashing pattern, color, etc. can be controlled. The vest also includes a battery 36 for powering the system and a charging 5V DC input 38 for receiving a cable for plugging into an outlet to charge the battery 36. An LED can be provided to indicate charging of the battery 36.

The vest is preferably made of a protective bulletproof material such as Kevlar to protect the first responder at the MCI. The vest pouch can be opened and medical supplies accessed while being worn by the wearer so the protective vest can provide protection while the first responder is at the site. The environmental sensors can also be read while the vest is worn, providing additional protection.

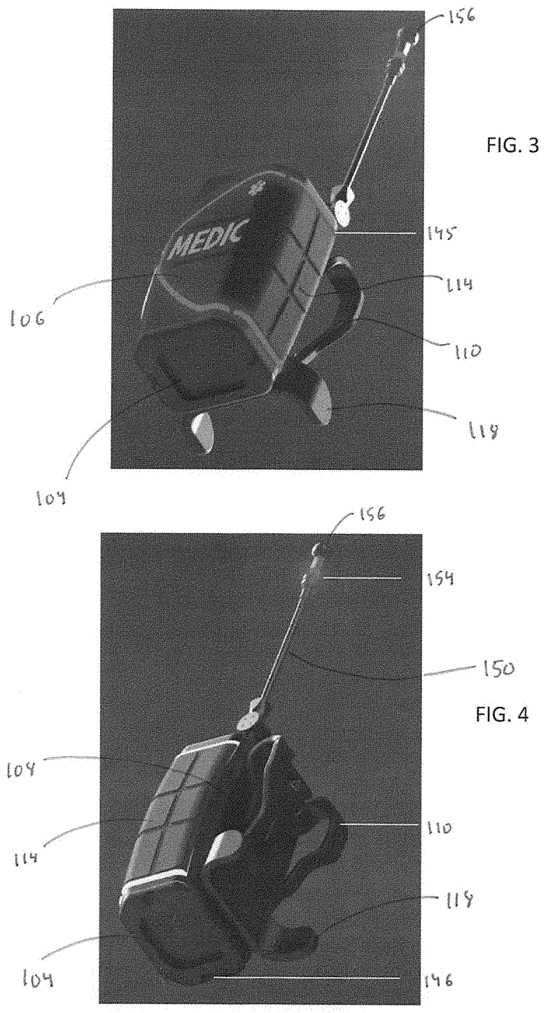

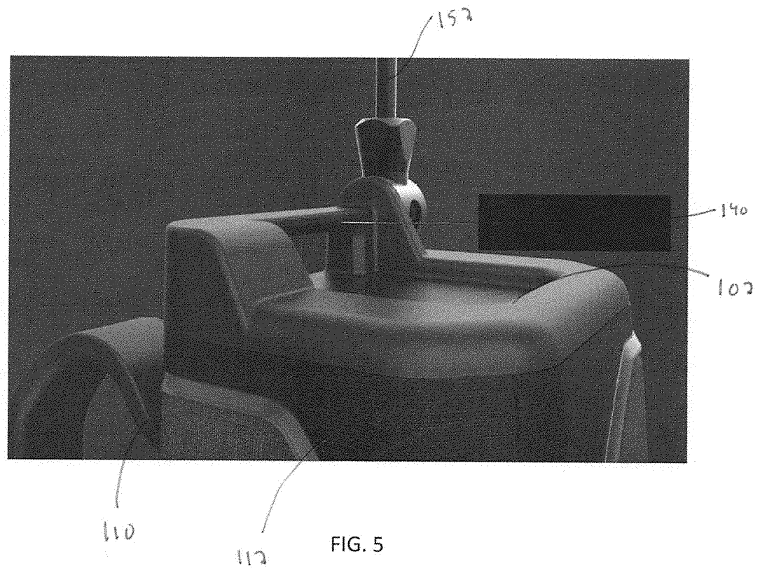

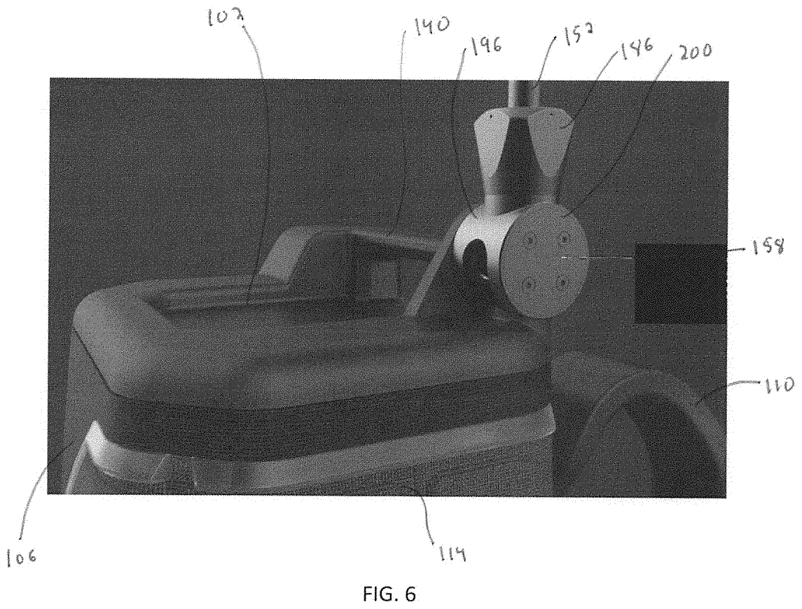

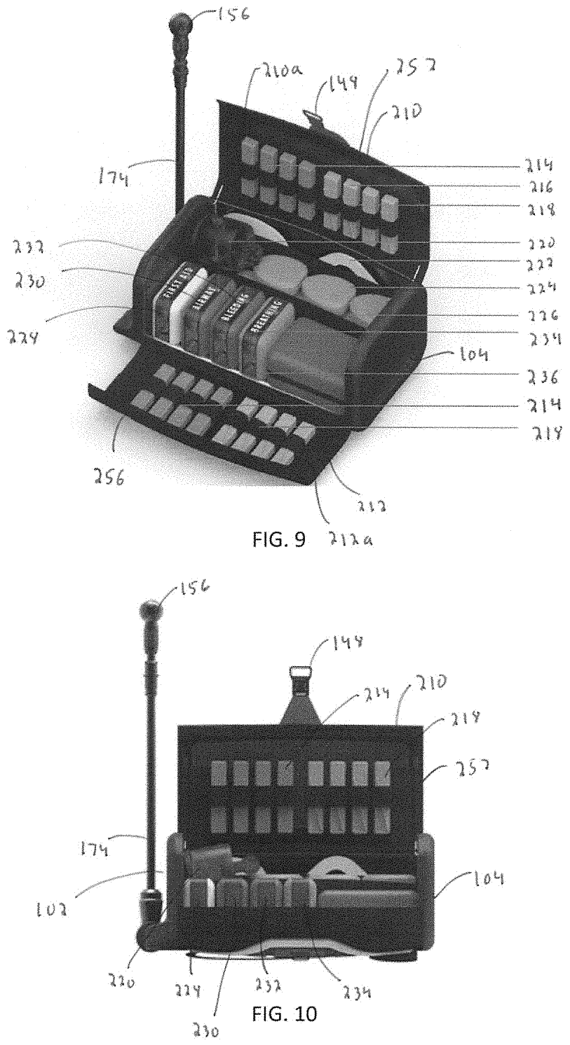

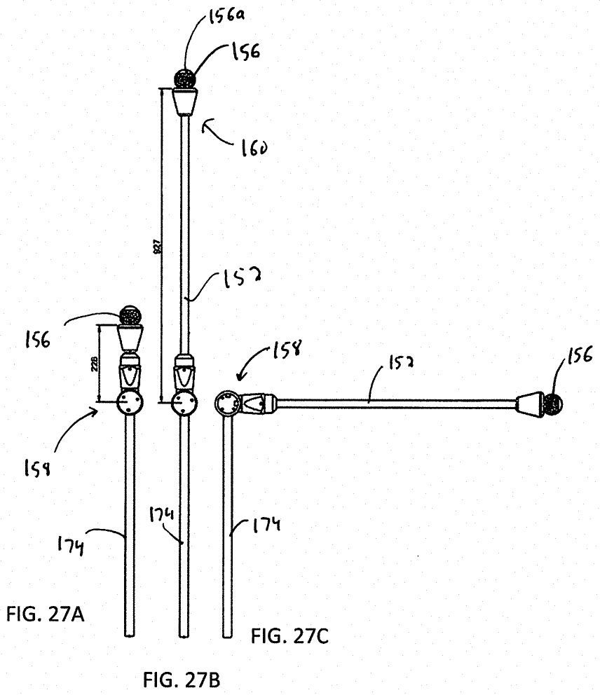

Turning now to the light weight portable smart bag (portable medical equipment carrying case) of the present invention, the smart bag 100, due to the electronics and construction described herein, provides organization for durability and quick medical item access with digital features that aid the responder in parting crowds, passively communicating with the teammates and recording the scene for command ops live use and post event playback. Smart bag 100 provides one example of a bag incorporating the electronics and features disclosed herein, and with initial reference to FIGS. 2-10, the bag 100 includes a beacon 150 which includes an extendable pole 152 having a beacon flashing light 154 and a 360 degree surveillance camera 156 mounted thereon. This makes the first responder visible to teammates and others while passively recording the event for live streams to ops and post event analysis. The pole 152 has a retracted position as shown for example in FIGS. 7 and 8 and an extended position as shown for example in FIGS. 3 and 4. The beacon 150 also has a rotatable feature 158 so that the pole 152 can be pivoted from a vertical position shown for example in FIGS. 3 and 4 to a horizontal position such as shown in FIG. 9. These extendable and rotatable features can be appreciated by comparing FIGS. 27A-27C wherein FIG. 27A shows the pole in the retracted position, FIG. 27B shows the pole in the extended vertical position and FIG. 27C shows the pole in the extended horizontal position. Components of the beacon and their function are described in more detail below.

FIGS. 2-10 illustrate various views of one embodiment of the bag of the present invention. The bag includes a top portion 102, a bottom portion 104, a front portion 106, a rear portion 108, a left side portion 112 and a right side portion 114. Extending from the top portion 102 is handle 140. The handle 140 aids in carrying the bag 100 in the vertical position. It can also be grasped in the horizontal position of the bag, with the carrier's other hand on the bottom panel to transport the bag in the horizontal position. Flashing lights 145 are provided on the panels, which in some embodiments can flash blue and red, indicating the wearer's identity as a first responder and allowing for easier crowd parting and patient access. The MEDIC insignia can be made of a reflective fabric.

The bag 100 also includes at the rear portion 108 a pair of straps 110. An overlapping strap contains the actuation buttons 10, 12, 14, 16 and 18 to activate the various components of the system. The button configuration, along with the accommodation for the wiring within the strap, is discussed in more detail below. The straps can also include front facing lights. A single hand clasp 148 opens the front and side panels as also discussed in more detail below. Speakers 146 for the siren can be provided on the bottom portion 104.

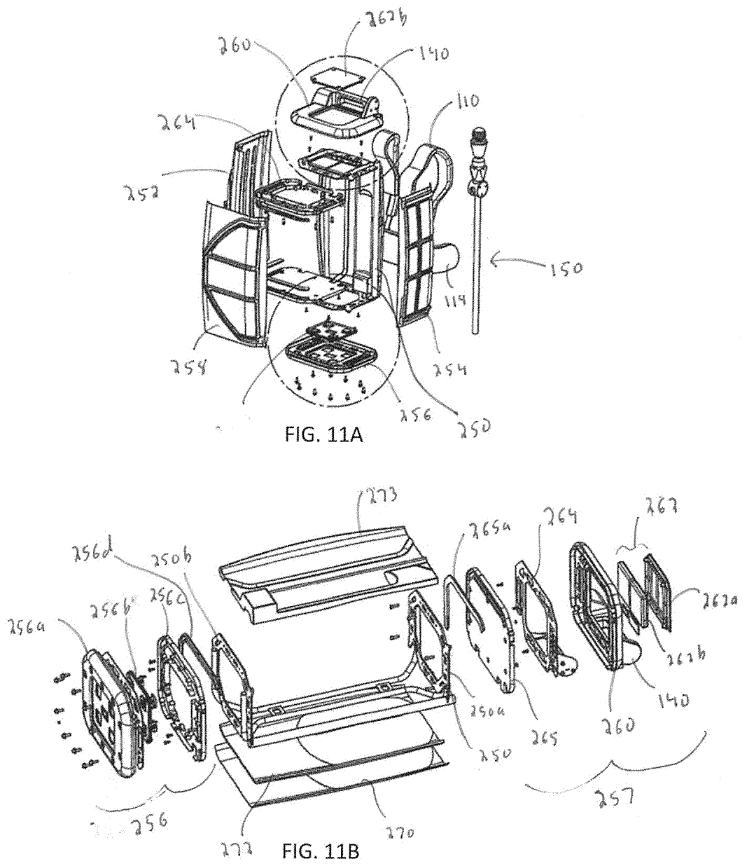

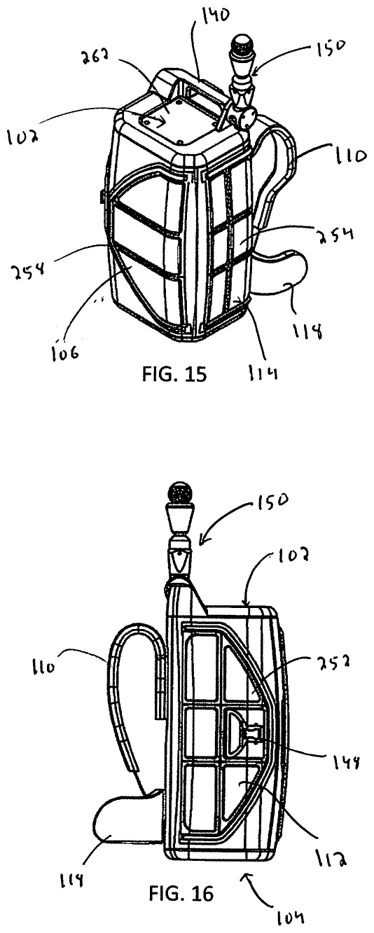

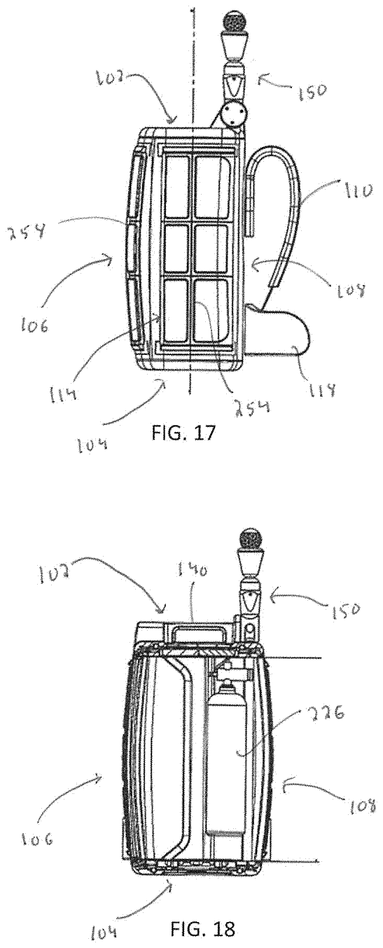

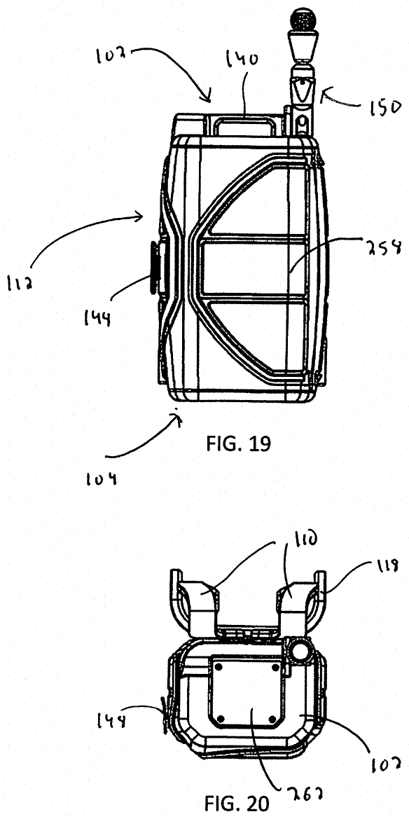

The construction of the panels which make up the portions of the bag 100 is illustrated in FIGS. 11A-20. This construction provides a lightweight transportable bag with sufficient strength and durability to retain the internal components and keep the bag open during use, i.e., the bag can be set down and the flaps (panels) when opened in a tool-box like fashion remain in the open position (see e.g., FIG. 9). Thus, the bag construction reduces tearing or other damage to the bag during use, while being sufficiently lightweight to minimize fatigue as the first responder moves from person to person in the triage environment at the multiple-casualty incident. The bag 100 also includes a fabric outer layer to provide the tactile feel of a bag for the user's familiarity. The bag without medical supplies in preferred embodiments can have a weight of about 25 pounds and with a full supply of medical equipment could have a weight of about 45 pounds. Other weights (empty and full) are also contemplated.

Turning first to the exploded views of FIGS. 11A and 11B and the assembled views of FIGS. 17-20, the bag 100 includes a rear frame 250 (at the rear portion 108), a left side panel 252 (at the left side portion 112), a front panel 258 (at the front portion 106), a right side panel 254 (at the right side portion 114) a bottom panel 256 (at the bottom portion 104) and a top panel 257 (at the top portion 102).

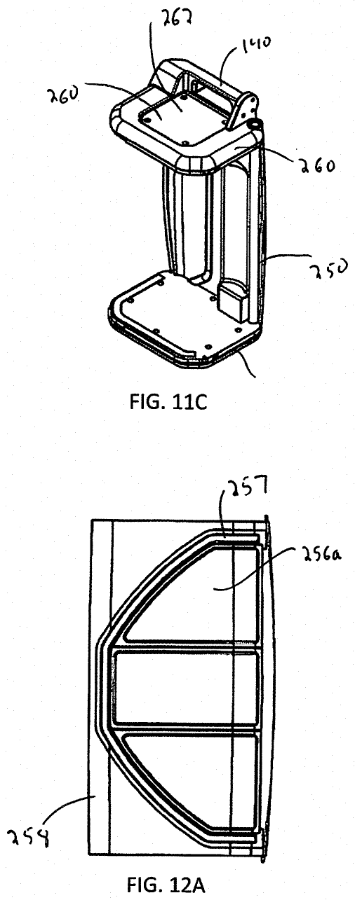

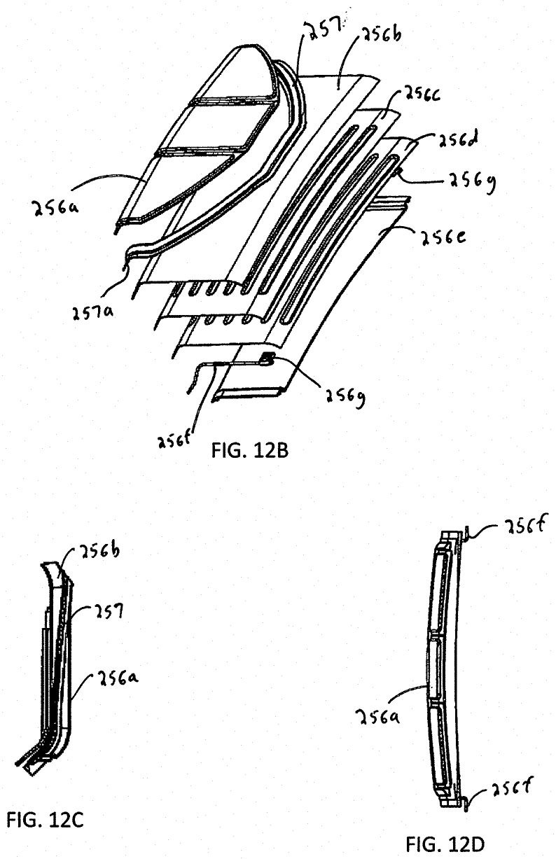

The front panel 258 and side panels 252, 254 in preferred embodiments are multilayered to achieve the durable lightweight construction discussed herein. The front panel 256, shown in detail in FIGS. 12A-12D, includes from an outermost to an innermost layer: a) a nylon outer panel 256a with contours for high density foam; b) a second layer 256b of nylon fabric; c) a third layer 256c of PET plastic with corrugations which can be vacuum formed for increased rigidity; d) another corrugated nylon layer (a fourth layer) 256d of the same material as the second layer 256b; and 3) an innermost layer 256e which has an internal Velcro fabric surface to hold components within the bag 100. The front panel 256 can further include an elongated member such as rod 256f at the upper and lower portions (as viewed in the vertical position of FIG. 11A), with an attachment 256g, which forms a hinge for opening the front panel 256 to access the contents of the bag 100. That is, the front panel 256 pivots about the hinge to open the bag 100, and the panel once opened (see e.g. FIG. 9) remains in this open position by rods 256f. More specifically, when the bag 100 is removed from the wearer's back and placed in the horizontal position on the ground, and the strap 148 unclasped, the front panel 256 is pivoted to an open position to reveal the contents of the bag 100 as well as to display the components attached, e.g., via Velcro, to the innermost layer 256d. This pivoting of the front panel 256a to the open position is shown in FIG. 9, and the contents of the bag 10 will be discussed in more detail below.

A tubing 257, which can be composed of PET for example, forms a light guide for the LED strip and as shown is an arcuate configuration, e.g., C-shaped, extending along the periphery of outer layer 256a. In this way, a light is emitted along the front panel, the light being either a static or blinking light. The tubing 257 can be attached to layer (panel) 256b by various methods. One method by way of example is flange 257a is sewn onto the fabric of second layer 256b.

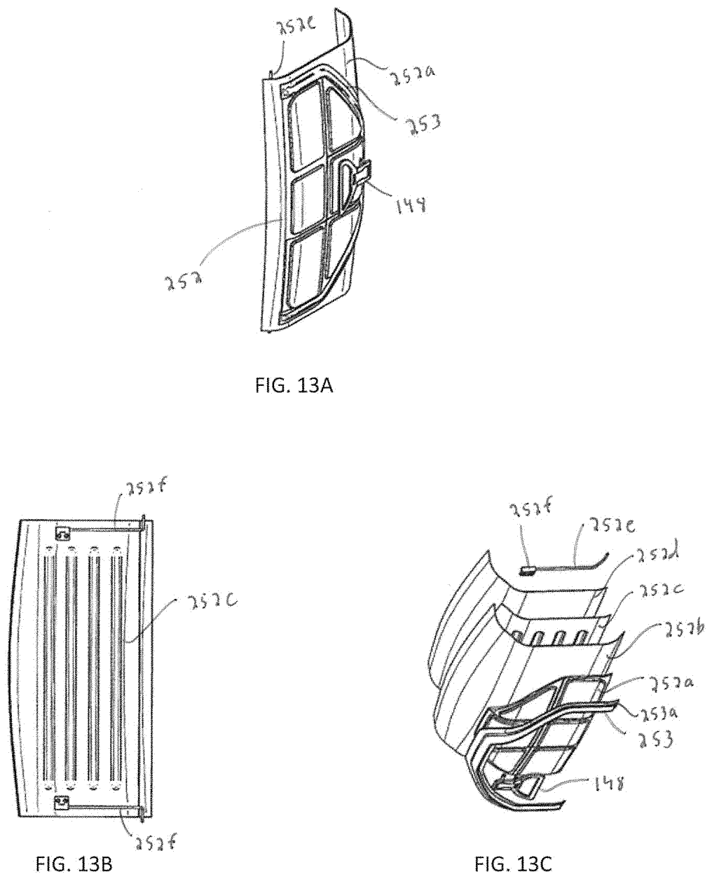

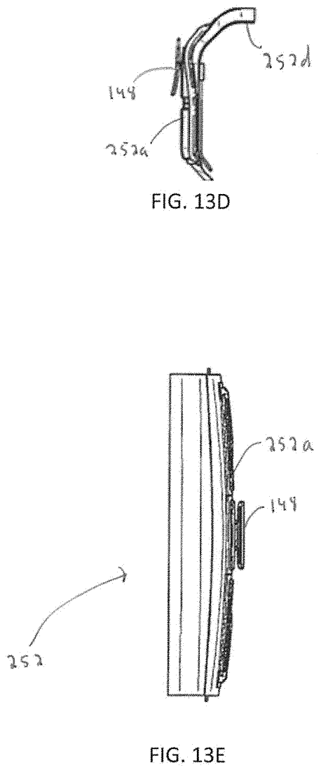

The left side panel 252 is the other openable panel of the bag 100. With reference to FIGS. 13A-13C, the left panel 252 includes from an outermost to an innermost layer: a) a nylon outer panel 252a with contours for high density foam; b) a second layer 252b of nylon fabric; c) a third layer 252c of PET plastic with corrugations which can be vacuum formed for increased rigidity; and d) an innermost layer 252d which has an internal Velcro fabric surface for holding components on the surface. The left side panel 252 further includes an elongated member such as a rod 252e at the upper and lower portions (as viewed in the vertical position of FIG. 11A), with an attachment 252f, forming a hinge for opening the left side panel 252 to display and access the contents of the bag 100. As in the front panel 258 discussed above, the rods 252f maintain the left side panel in the open position of FIG. 9 when the left side panel 252 is pivoted to the open position about the hinge when the bag 100 is removed from the back of the wearer, placed in the horizontal position and the clasp 148 unclasped. This pivoting of the front panel 252 to the open position is shown in FIG. 9, and as can be seen components, e.g., tourniquets, are attached via Velcro to the inner fabric surface of innermost layer 252d. Note the left side panel 258 also includes the clasp 148 which in preferred embodiments provides a quick release single hand clasp so the wearer can release the clap to easily open and reclose (reseal) the bag with one hand. These embodiments of the single hand clasp, when utilized in the bag 100 of the present invention, can in certain circumstances be less prone to mechanical failures and increased opening time associated with zippers and buckles. However, it should be understood that other release mechanisms including zippers and buckles can be utilized in alternate embodiments and might be beneficial in certain circumstances.

A tubing 253, which can be composed of PET, forms a light guide for the LED strip on the left side panel 253a and as shown has an arcuate configuration, e.g., C-shaped, extending along the periphery, e.g., following the contours, of outer layer 252a. It can be attached to panel 252 by various methods. One method by way of example is flange 252a is sewn onto the fabric of second panel 252b.

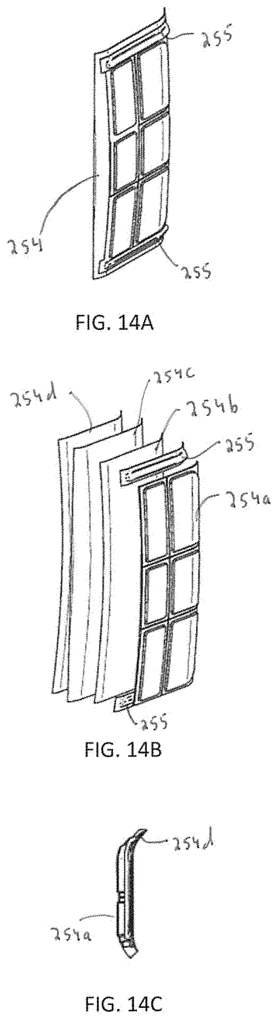

The right side panel 254 is shown in more detail in FIGS. 14A-14c and unlike the left side panel 254 does not open. The right panel 254 includes from an outermost to an innermost layer: a) a nylon outer panel 254a with contours for high density foam; b) a second layer 254b of nylon fabric; c) a third layer 254c of PET plastic; d) and an innermost layer 254d. The right side panel 254 includes a PET light guide 255 at the upper and lower portions (as viewed in the vertical position of FIG. 11A), each extending transversely as shown to provide lighting, either blinking or static, across the top and bottom of the right side panel 254. The light guides 255 can be attached to the outer layer 254a by being sewn to the fabric, although other attachment methods are also contemplated.

Note the light guides on the panels can be positioned at other locations other than those shown and can also be other shapes and configurations. Also, a different number of light guides than the number shown in the drawings can be provided.

The rear frame 250 shown in FIGS. 11B and 11C includes a rectangular top support 250a onto which is mounted a multilayered panel 257 composed of from the outermost to the innermost layer a) a cover 262 having a rectangular frame support 262a and a top cover panel 262b; b) a handle support 260 from which handle 140 extends (either integrally/monolithically or as a separate attached unit; c) top open frame 264; and d) an inner panel 265. A light guide 265A which can be L-shaped as shown is attached internally of top support 250a. Attached to the rectangular bottom support 250b of rear frame 250 is multilayered bottom panel 256 composed of from the outermost to the innermost layer a) bottom cover 256a, b) an inner panel 256b; and c) a bottom open frame 256c. Attached to an inner surface of bottom support 250b is a light guide 256d which can be L-shaped as shown. Light guides 256d and 265a provide interior light for the bag when the front and side panels are open. Note the components of the bag 100, e.g., the panels, supports, etc., can be attached for example by riveting, although other methods/fasteners are also contemplated.

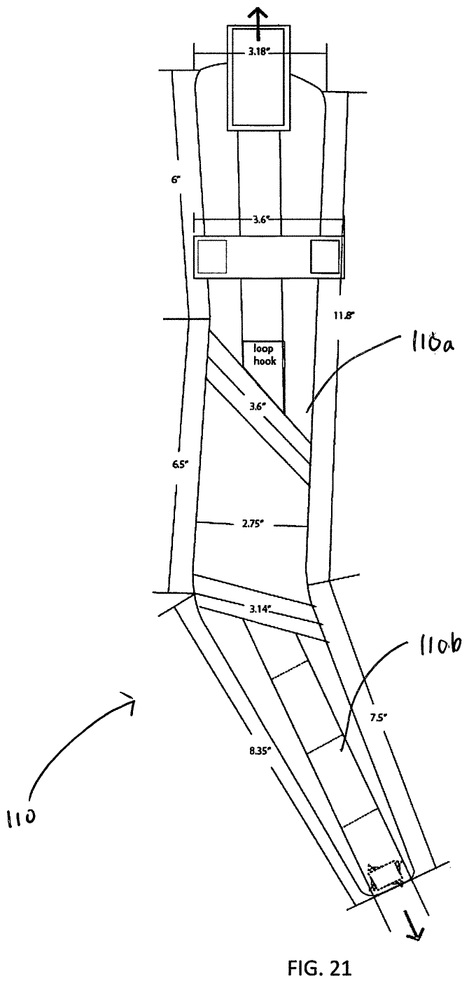

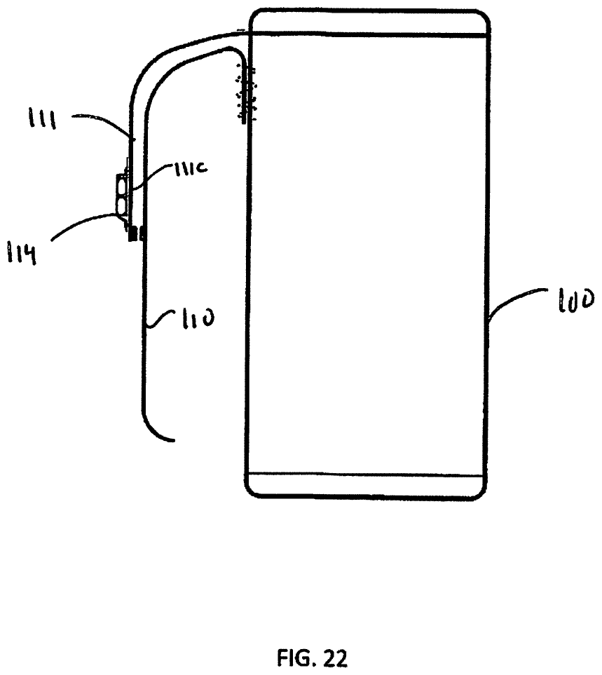

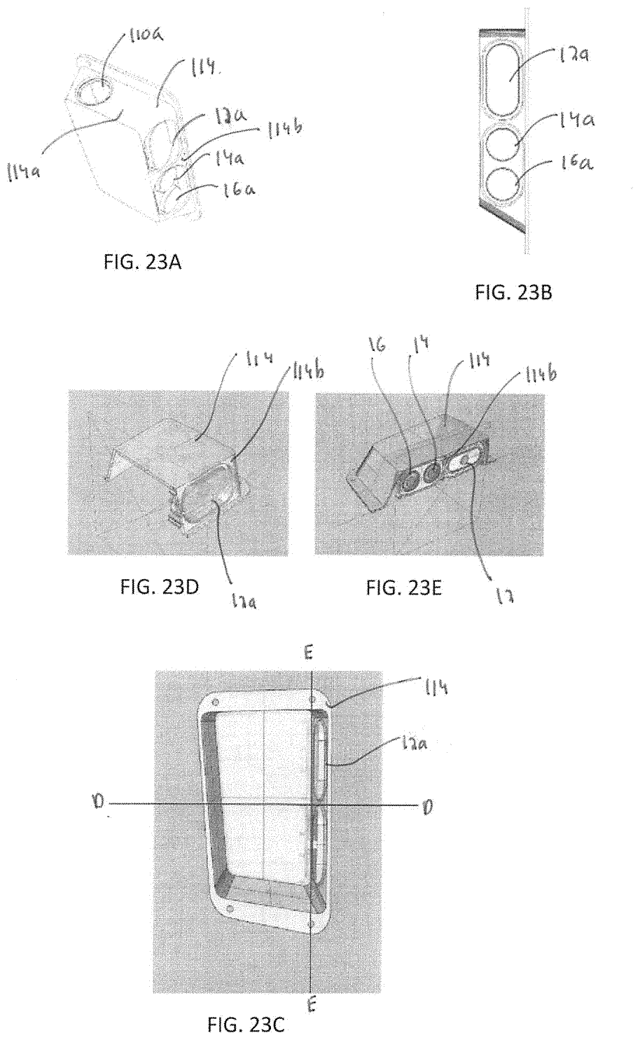

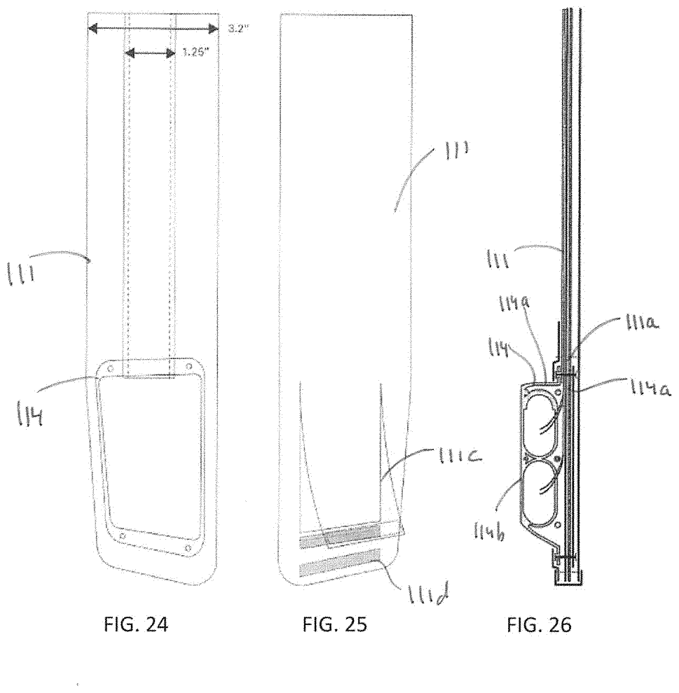

Turning now to the straps 110 of the bag, and with reference to FIGS. 21-26, the pair of straps 110 are for supporting the bag 100 on the wearer's shoulders/back. A secondary external strap 111 supports the buttons for actuating the electronics and includes a channel for the wires connecting the buttons to the LED and siren components carried/supported by the bag 100. As shown in the side view of FIG. 21, the strap 110 has a vertical portion 110a and an angled lower portion 110b. Each strap 110 is connected to the rear panel of the bag at a top and bottom portion such as by stitching to the bag or by other methods of attachment. The straps 110 form an “inner strap” of the bag 100. A second strap 111, shown schematically in FIG. 22, is positioned over one of the straps 110, but spaced apart as shown. The strap 111 extends partially external of a length of a strap 110, and in the illustrated embodiment, terminates approximately midway of strap 110, although a strap 111 of other lengths are also contemplated. The strap 111 has an inner wall and an outer wall, thereby forming a channel 111a (FIG. 26) for passage of wiring 117 from button housing 114. In this manner, the wiring 117 can be kept separate from the bag strap 110 so that the button housing 114 and wiring 117 can be changed without interfering with the construction of the straps 110 and bag 100. This provides increased modularity of the system. Button housing 114 is positioned on an external portion of the strap 111, preferably at a lower portion of the strap 111, as shown in FIG. 22. Button housing 114 includes the power button receptacle 10a for power button 10 which turns on the system and camera, button receptacle 12a for button 12 which turns on the LED and siren, and smaller button receptacles 14a, 16a for buttons 14 and 16 which turn on respectively the LED and the siren. In the illustrated embodiment, the power button 10 is along one surface 114a of the housing and the other three buttons 12, 14 and 16 are along another surface 114b, the surface 114a being a top facing surface and the surface 114b being a side facing surface transverse to the top surface 114a.

A flap 111c (FIG. 25) on the back (rear) side of the strap 111 allows access to the wiring 117, and is openable as shown. It can be maintained in a closed position by Velcro 111d. The button housing 114 can be riveted to the strap 111, although other methods of attachment are also contemplated. The wires 117 extend though the channel 111a into the bag for connection to the camera, lights and siren as described above and depicted in the flow charts showing the user interface and electronics of the system.

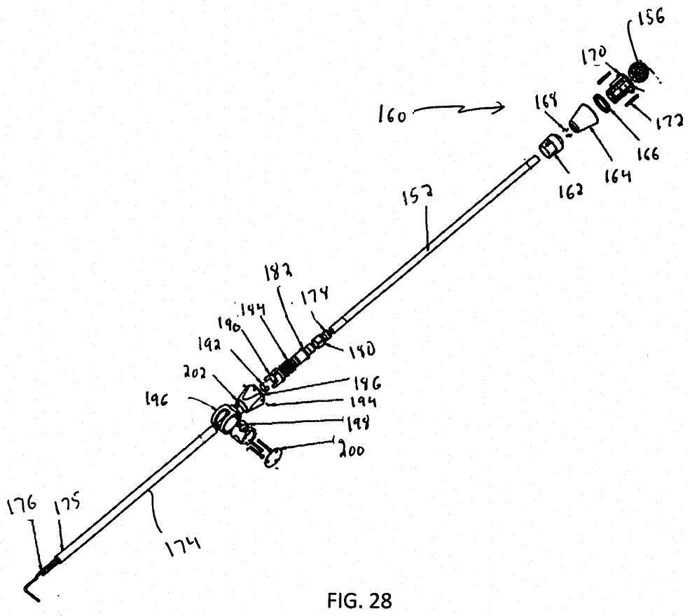

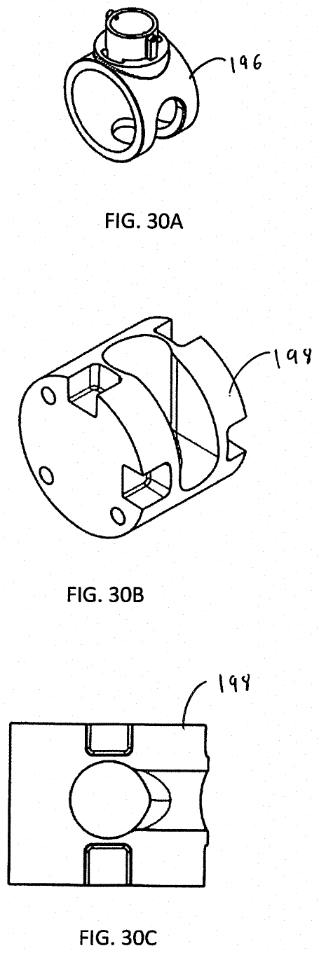

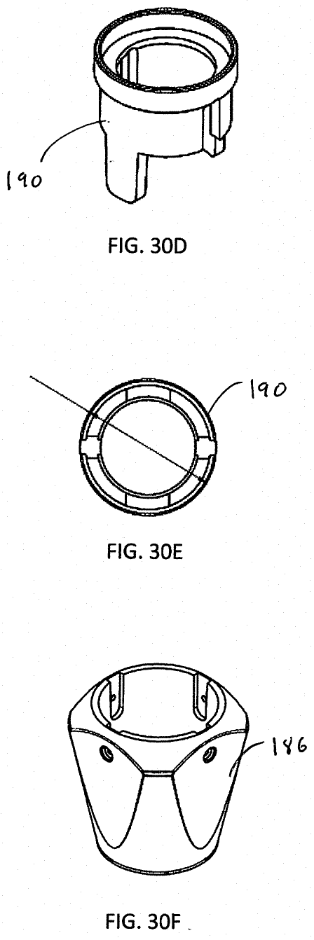

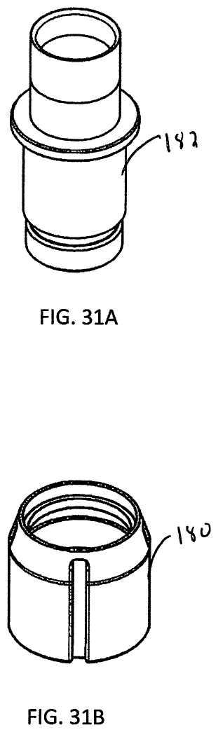

Turning now to the beacon 150, FIGS. 27A-27C as mentioned above, illustrate the three positions (orientations) of the extendable pole 152 of the beacon: 1) retracted and vertical (not rotated) shown in FIG. 27A; b) extended and vertical (not rotated) shown in FIG. 27B; and extended and horizontal (rotated) shown in FIG. 27C. Details of the beacon 150 are shown in FIGS. 28-31B and will now be described.

At the proximal top end of the beacon 150, referred to as the top portion 160, are light cover 164, neopixel ring 166, a camera base 170 with an extension 173 and a post 171 for mounting camera 156. Camera 156 is preferably a 360 degree camera to provide full surveillance at the MCI site. Strips 172 are positioned around the perimeter of conical light cover 164. In the illustrated embodiment, four strips 172 are provided around a 360 degree perimeter of the cover 164, and each strip 172 can have for example eight lights, e.g., LEDs, along their lengths, although a different number of lights within each strip and a different number of strips can be provided. The lights are preferably blinking lights that are turned on when the system is turned on by button 10. These flashing lights at the top of the beacon 150 indicate the presence of the first responder and facilitate clearing/parting crowds in an ambulance or emergency-like fashion.

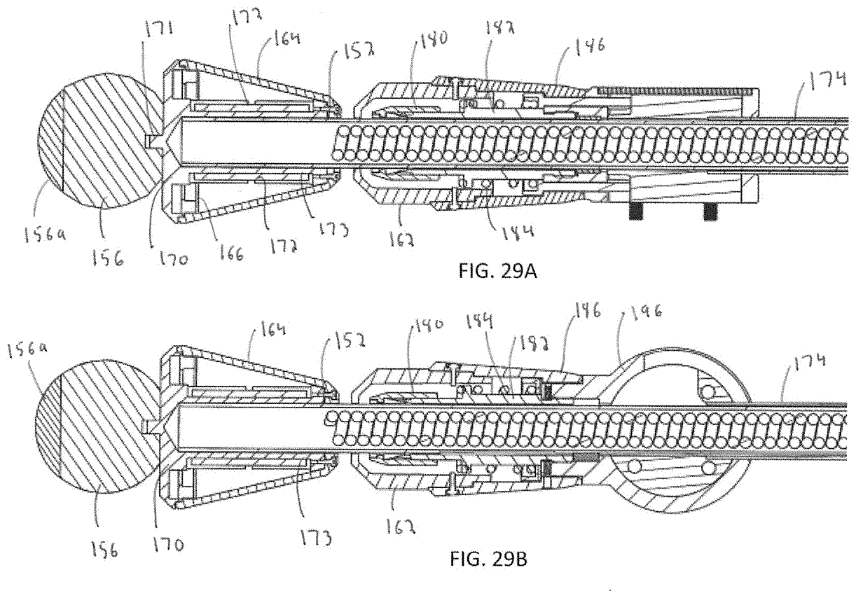

The retractable pole 152 is telescopingly received in outer tube 174 for slidable movement therein from an initial retracted position to an extended position. Release top 162 is mounted to release base 186 and as shown in the cross-sectional views of FIGS. 29A and 29B, the distal portion 162a is riveted to the proximal portion of release base 186. In this position of FIG. 29A, the pole 152 is maintained in the retracted position.

Tube clamp 178 clips into boom lock 180 and this assembly screws onto boom base 182 with center tube (pole) 152 in place. The screwing action jams (compresses) tube clamp 178 and boom base 182 together. Tube clamp 178 has a taper on the inside, thus forcing tube clamp 178 closed to clamp on center pole 152 to hold the pole 152 in place. Such clamping is combined with the swivel lock in one hand operation. With boom lock 180 having slots on the side, as it is screwed for the clamping action of pole 152 it moves up and down inside the handle assembly Release top 162 has matching grooves on the inside that make boom lock 180 rotate.

The rotatable mechanism 150 for pivoting (swiveling) the pole to a 90 degree angle includes a rotation key 190, a tube key 192 and a release base 194. Spring 184, rotation key 190, and tube key 192 are sandwiched in boom base 182. Fixed beacon 198 is positioned within the opening into rotator 196. Cover 200 is seated over fixed beacon 198. The assembly with boom base 182 can be pulled up with a spring action and as a result unlocks the rotation key 190 from the fixed beacon 198. When pulled up to this release position, the assembly can be rotated 90 degrees. Tube nest 174 acts as a stop (at its proximal end) to prevent the assembly from rotating past 90 degrees which could damage the cable. Pole 152 has a groove that aligns with the protrusion on tube key 192 that prevents pole 152 from rotating and thus protecting the cable, The release top 162 has matching grooves on the inside that make boom lock 180 rotate. Clamping of the sliding pole 152 with the 90 degree lock swivel action of the pole 152 is achieved with a single hand operation.

Referring back to FIGS. 2-9, the foregoing components incorporated into one embodiment of a smart bag in its final assembled form can be appreciated. Lightweight ergonomic smart bag 100 is shown in FIGS. 7 and 8 with the pole 152 in the retracted position and FIGS. 2-4 show the smart bag 100 with the pole 152 in the extended position. FIGS. 9 and 10 show the smart bag 100 in the horizontal position with the front and side panels 252, 256 opened to reveal the internal contents of the smart bag (and trigger the internal light).

The bag 10 can contain various emergency kit supplies/components and FIGS. 9-10 show one example of the supplies. Other supplies can also be provided either in addition or as an alternative to any of the supplies illustrated herein. As can be appreciated, when the bag 100 is open, all the supplies can be visualized. Further, the medical supplies are packed in an organized fashion so the first responder can not only at a glance see each of the contents but can easily access the contents. Thus, the first responder does not need to dig through the bag or search separate compartments to locate the supplies, which can result in the loss of valuable time. By way of example, the components within bag 10 in FIGS. 9 and 10 are as follows: 1) tourniquets 214 mounted by Velcro on the front panel 252 and side panel 256; 2) trauma kits 218 mounted by Velcro on the front and side panels 252, 256; 3) sleeve 216; 4) bag valve mask (BVM) 220; 5) C-collar 222; 6) CPR mask with oxygen inlet and case; 6) oxygen tank 226; and 7) four organizer bags—first aid 228, airway 230, bleeding 232 and breathing 238; and 8) AED kit 236.

Thus, as can be appreciated, the lightweight and durable smart bag (transportable carrying case) 100 of the embodiments of FIGS. 2-10 includes a) a user interface comprising a plurality of switches on the strap selectively activated by the user, b) lighting features to provide external light on the bag; c) speaker(s) for the siren/alarm; d) a surveillance camera for data collection and storage/post analysis; d) openable flaps to provide easy access to medical supplies; e) internal lighting to light the bag when opened; and f) an easily accessible and conveniently organized optimized collection of medical supplies.

In some embodiments, the smart bag 100 can include a GPS chip so a central command can track the location of the first responder to maximize response to the injured at the MCI. In some embodiments, communication systems, e.g., WiFi communication, can be provided so the first responder can communicate with other first responders at the scene as well as with the central command. In some embodiment, the smart bag includes a long range radio chip and antenna that transmits to a designed homebase unit containing a WiFi router and receiving antenna. In some embodiments, the bag has a cellular module for wireless data transfer. In some embodiments, photo and video data are stored onto an SD card which is later removed to access surveillance content.

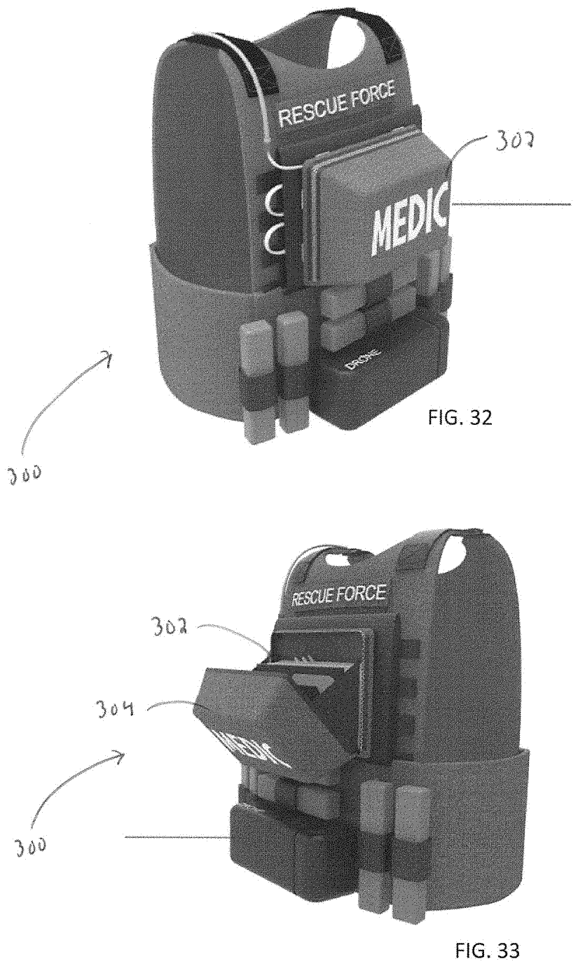

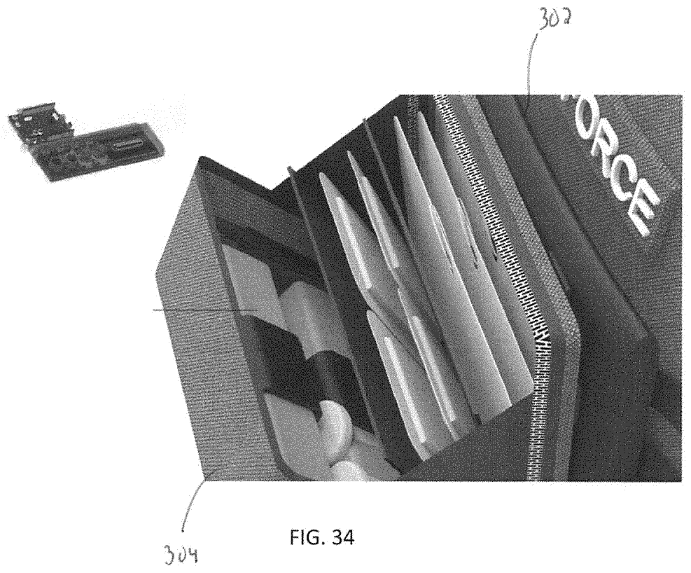

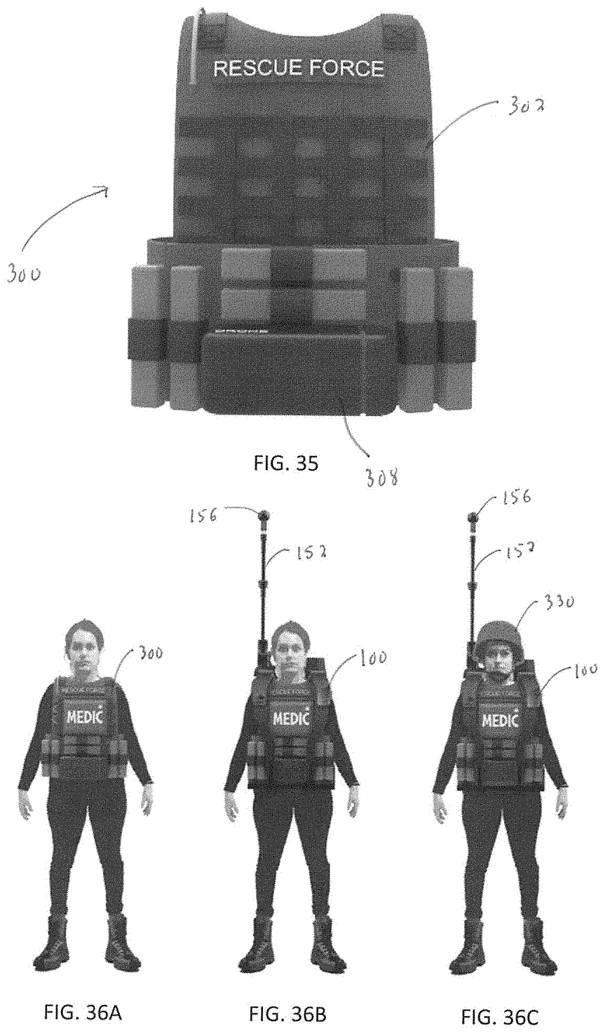

The vest 300, preferably bullet proof and made of Kevlar, can provide a supplement to the smart bag or alternatively be used independently. The vest 300 as described in detail above has an environmental (air quality) sensing unit and light. The vest 300, as shown in FIGS. 32-34 embodies one version containing the electronics discussed above. Vest 300 has a front pouch 302. In some embodiments, the pouch 302 can be removably mounted to the vest 300 to increase the modularity of the system so pouches containing varied supplies can be mounted to the vest 300 (FIG. 35 shows the Vest 300 with the pouch removed). The pouch 302 has a bottom hinged front panel 304 which is openable to view and access the contents while being worn by the first responder. A space for a drone can be provided to allow for a birds-eye view and scene recording for live and past-event responder use. Various supplies can be provided in the pouch. In one embodiment by way of example, the contents can include four tourniquets, two six inch trauma dressings, two rolled gauze, two nasopharyngeal airway with lubricant, a Hyfin* vent chest seal twin pack, three pairs of Nitrile gloves, a pair of trauma shears and scissors, four casualty cards, a mid ID patch and two rescue task force patches. Other supplies are also contemplated.

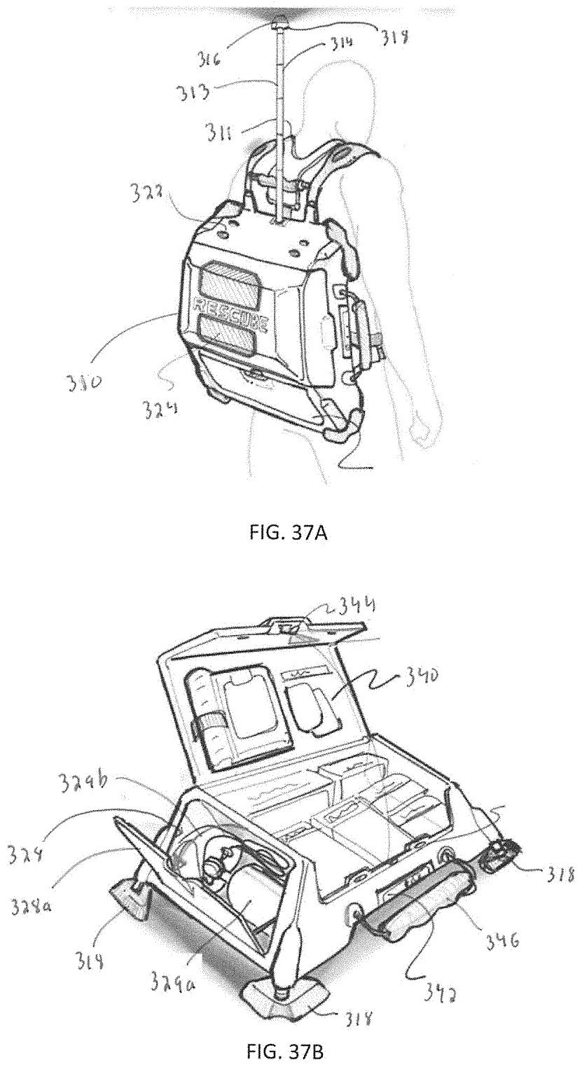

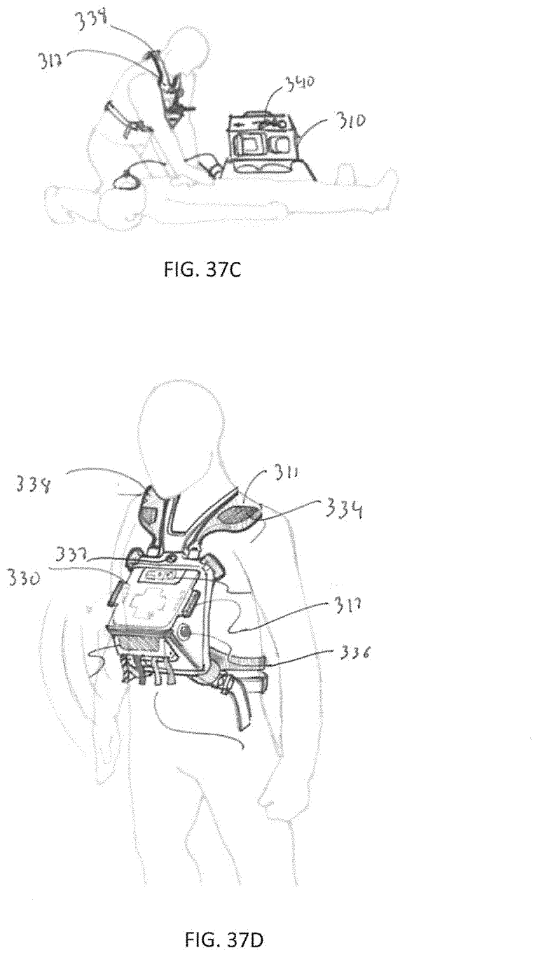

FIGS. 37A-37D illustrate alternate embodiments of the transportable (portable) medical supply (equipment) carrying case (smart bag) and vest of the present invention. Turning first to the embodiment of FIGS. 37A-37D, the bag 310 in the form of a backpack extends from the neck structure 311, carried on the wearer's back. Vest 312 extends from neck structure 311 and is carried in the front of the wearer. The bag/backpack 310 and/or the vest 312 can be integral with the neck structure 311 or alternatively a separate unit attached to the neck structure 311 so they can be independently removed. Bag 312 includes a beacon 313 similar to beacon 150 of FIG. 2 in that it has an extendable pole 314 with a surveillance camera 316 and flashing light (LED) 318 at the top. Modular snap on 322 atop bag 310 enables attachment of a case containing additional medical supplies. One or more speakers 324 are positioned on the rear portion of the bag 310. An oxygen tank 329a and oxygen mask 329b are contained in bottom compartment 328, which includes a hinged door 328a openable as shown in FIG. 37B.

The vest 312 contains a case or storage unit 330 which can contain a first aid kit and/or other medical supplies. A surveillance camera 332 is positioned adjacent a top portion of the vest 312. Vest 312 can also include a speaker 334 for the alarm/siren. Actuation button 336 is provided on vest 312. The straps 338 of the vest structure 311 can include sensors such as air quality sensors to provide readings of the air at the site to check for the presence of hazardous gasses as in the aforedescribed embodiments.

The bag 310 in use is removed from the wearer's back and placed in a horizontal position as shown in FIGS. 37B and 37C. The bag 310 includes four adjustable legs 318 which support the bag 310 in the horizontal position. In some embodiments, the legs can be adjusted to change the height of the bag 310 with respect to the ground or other surface on which the bag 310 is positioned. The back panel 340 is hingedly connected to the bag and is openable to display the internal components, i.e., the medical supplies, contained within the central compartment of the bag 310. The back panel 340 can also be used to hold supplies such as a defibrillator. The back panel 340 can in some embodiments be maintained by magnetic locks 342 on the bag 310 interacting with locking tab 344 on the back panel 340. Side handle 346 aids in transport of the backpack 310. The bag 310 (and bags 350 and 380 described below) can include the electronics and lights described above, e.g., turn on when the bag is open and off when the bag is closed.

The bag 310 (and bags 350 and 380) can include multilayered panels as in FIGS. 11A-14C. The bag 310 (and bags 350 and 380) can have an auxiliary strap for the wires and actuator as in FIG. 22.

In use, the bag 310 is removed from the wearer's back and the vest 312 remains worn by the user. Bag 310 is placed horizontally on the ground as shown in FIG. 37C and the back panel 340 (now the top panel in the horizontal position of the bag) is opened and remains in the open position. The supplies are visible when the back panel 340 is opened to display all the contents. If oxygen is needed, the bottom panel 341 (now the side panel in the horizontal position of the bag) is opened to access the oxygen tank 329a and mask 329b.

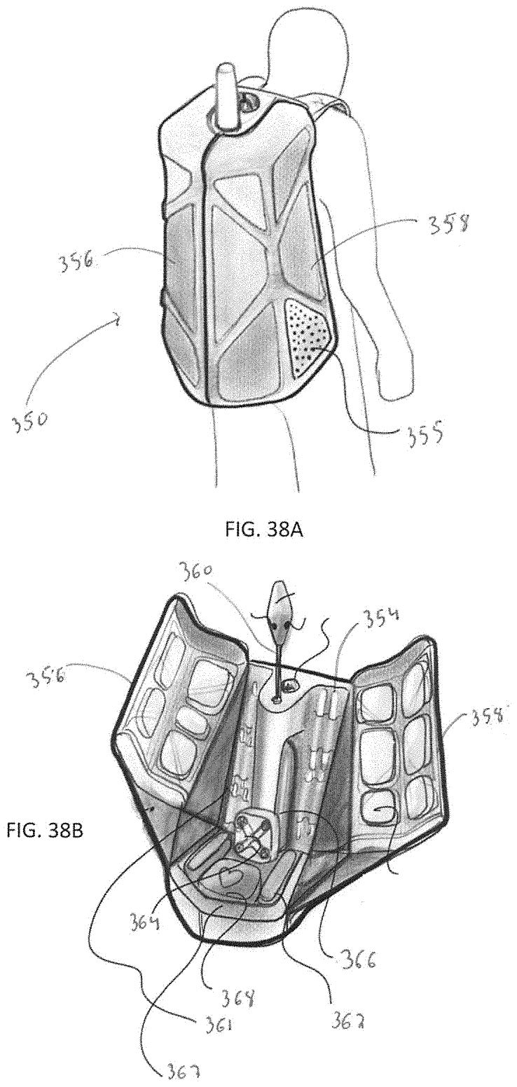

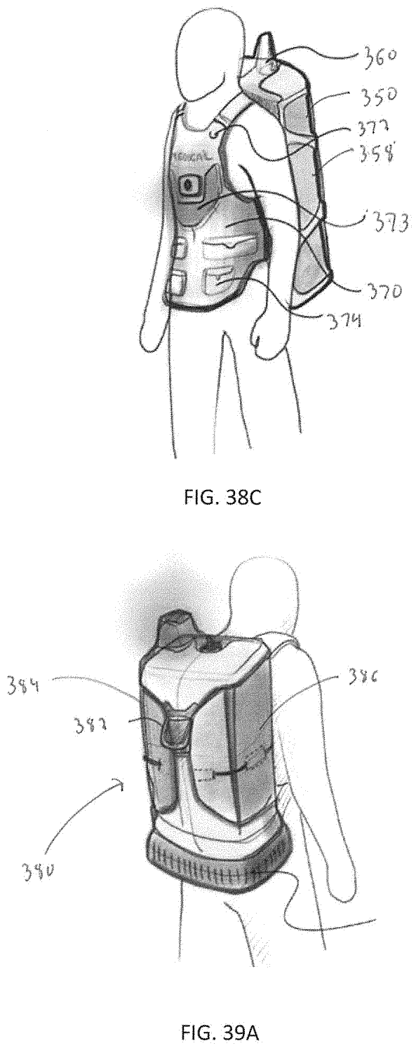

The bag of the alternate embodiment of FIGS. 38A-38C is openable in a different manner. The bag 350 is worn on the back in a backpack like fashion and is formed from a rear panel 354, a left side panel 356 and a right side panel 358. A siren/alarm 355 can be provided on one of side panels 356, 358. Bag 350 is removed from the wearer's back (with the vest 352 remaining on the wearer) and placed in an upright position on the ground. Side panels 356 and 358 are opened in a bat-like manner (or wing like fashion) to display the internal contents. The side panels 356, 358 can include Velcro for attaching supplies to the inside of the panels 356, 358. The rear panel 354 can include sleeves 361 for medical tools or supplies. The bag 350 also includes an extendable pole 360 with a camera and LED as in the embodiment of FIG. 2. The bag 350 contains batteries 362 to power the LED and siren. The bag 350 can also include a drone 364, an oxygen tube 366, and a defibrillator 368. The bag 350 can also include a gusset to enlarge the surface. A Kevlar vest 370 can also be provided having a button 372 to activate a siren/alarm and/or flashing light 373 on the front of the vest 370. The vest can include pockets 374. The bag 350 and/or vest 370 can be attached to neck structure 375 for removal or the bag 350 and/or vest 370 can be integral with the neck structure 375.

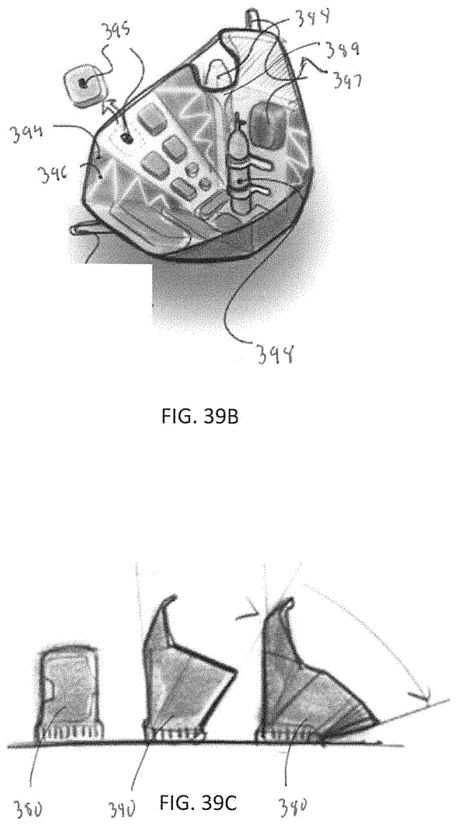

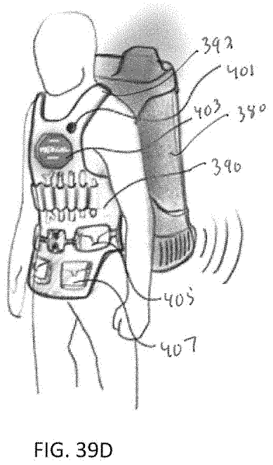

In the alternate embodiment of FIGS. 39A-39D, bag 380 has a pull handle 382 to open the left and right side panels 384, 386, respectively. The bag 380 opens in the manner shown in FIG. 39C with the first drawing depicting the bag 380 in the closed position, the second drawing depicting the bag 380 in the half open position and the third drawing depicting the bag 380 in the fully open position. In the fully open position of FIG. 39B, the contents of the bag 380 are fully visible and the bag 380 opens into a somewhat cup shaped configuration. A vest 390 extends from neck structure 392. The bag 380 and/or vest 390 can be attached to neck structure 392 for removal or the bag 380 and/or vest 390 can be integral with the neck structure 392. Bag 380 can include a beacon with an LED 388 mounted atop an extendable pole 389 as in the FIG. 2 embodiment. The bag 380 can also include a gusset 394 to enlarge the surface, one or more light pipes 396 to illuminate the inside of the bag, and built in Velcro 395 for easy attachment/detachment of medical supplies. A defibrillator 397 and oxygen tank 398 can be contained within the bag 380. The vest 390, as in the other vests disclosed herein, can be bulletproof and made of Kevlar. Vest 390 includes a camera 401, a front light 403, a utility belt 405 and a series of pockets 407.

The bags of FIGS. 37A-39C can include the various features/electronics of the bag of the embodiment of FIG. 2 and can carry the various medical supplies disclosed herein. The vests of FIGS. 37A-39C can also include the various features, e.g., sensors, and electronics of the vests described herein such as vest 300 of FIG. 32. The vest of FIG. 37A-39C can also carry the various supplies disclosed herein. The bags of FIGS. 37A-39C are easily openable to display in an organized and easily viewable (e.g., all contents are shown when open) and easily accessible fashion the medical supplies contained in the bag. The sirens and flashing lights help clear crowds in the manner described herein as the systems of FIGS. 37A-39C also can be considered as providing “portable ambulances.”

Although the apparatus and methods of the subject invention have been described with respect to preferred embodiments, those skilled in the art will readily appreciate that changes and modifications may be made thereto without departing from the spirit and scope of the present invention as defined by the appended claims.

What is claimed is:

1. A portable ambulance comprising:
a siren for crowd parting;
a flashing light, the flashing light mounted to an extendable pole;
a portable medical equipment carrying case, the extendable pole mounted to the carrying case and extendable with respect to the carrying case; and
a camera mounted to the extendable pole, the extendable pole movable from a retracted position wherein the camera is closer to the case and an extended position wherein the camera is further from the case.

2. The portable ambulance of claim 1, wherein the system includes an activation switch, wherein the activation switch enables activation of the siren.

3. The portable ambulance of claim 1, wherein the system includes an activation switch, wherein turning on the activation switch automatically turns on the camera.

4. The portable ambulance of claim 1, wherein the system includes an activation switch, wherein the activation switch enables activation of the siren and activation of the flashing light, wherein upon turning on the activation switch, the user can select one of only turning on the siren, only turning on the light or turning on the siren and light simultaneously.

5-6. (canceled)

7. The portable ambulance of claim 1, wherein in the extended position the extendable pole is rotatable from a vertical orientation to a horizontal orientation.

8. The portable ambulance of claim 7, further comprising a releasable locking mechanism, the locking mechanism releasable to enable rotation of the extendable pole.

9. The portable ambulance of claim 1, wherein the case has first and second panels, the panels openable when the case is placed in a horizontal position.

10. (canceled)

11. The portable ambulance of claim 1, wherein the system includes a flashing light on the case, wherein when the case is opened the flashing light either a) automatically changes to a static light or b) automatically turns off.

12-13. (canceled)

14. The portable ambulance of claim 9, wherein the first and second panels include a Velcro surface to hold medical supplies.

15. The portable ambulance of claim 1, further comprising a communications system for communication to a control center.

16. (canceled)

17. A portable ambulance comprising:
at least one blinking light;
a siren; and
a portable medical equipment carrying case, the flashing light and siren attached to the carrying case, the carrying case having a) a vertical position for wearing by a user on a back of the user and b) a horizontal position when removed from the wearer, wherein in the horizontal position a rear panel of the carrying case is positionable parallel to a support surface, and a front panel is pivotable to an open position transverse to the rear panel and a side panel is pivotable to an open position more aligned with the rear panel;
wherein upon opening the case, the at least one blinking light automatically changes its blinking status.

18. The portable ambulance of claim 17, wherein the status of the blinking light changes to a static light.

19. The portable ambulance of claim 17, wherein the status of the blinking light changes to an off position.

20. The portable ambulance of claim 17, wherein upon closing the front and side panels, the light automatically reverts back to its blinking status.

21. The portable ambulance of claim 17, wherein the front panel is composed of multilayered materials including a plastic interposed between two layers of nylon material.

22. The portable ambulance of claim 17, further comprising a light guide supported on the front panel to provide the blinking light, the light guide having an arcuate configuration and following the contour of an outer layer of the front panel.

23. (canceled)

24. The portable ambulance of claim 17, wherein the front panel comprises first and second rods at top and bottom portions providing hinges for pivoting the front panel.

25-26. (canceled)

27. The portable ambulance of claim 17, further comprising a beacon having an extendable pole, extendable with respect to the carrying case, and a flashing light and camera are positioned at a top region of the extendable pole, the pole pivotable from a vertical position to a horizontal position.

28. (canceled) a flashing light and camera are positioned at a top region of the extendable pole, the pole pivotable from a vertical position to a horizontal position.

28. (canceled)

29. A portable ambulance for treating patients at a multi-casualty incident site and simultaneously passively gathering data, the portable ambulance comprising;
at least one blinking light;
a siren;
a surveillance camera;
a light weight portable medical equipment carrying case, the flashing light and siren attached to the carrying case, the carrying case having a) a vertical position for wearing by a user on a back of the user and b) a horizontal position removed from the wearer, the carrying case having an openable first panel and an openable second panel, wherein the first and second panels are composed of multi-layers of material.

30-31. (canceled)

32. The portable ambulance of claim 29, wherein in the horizontal position, the first and second panels are openable in opposite directions to display medical supplies contained within the carrying case.

33-47. (canceled)
